(19) United States
(12) Patent Application Publication
(10) Pub. No.: US 2024/0306571 A1
(43) Pub. Date: Sep. 19, 2024

(54) PRECISION METERING AND DELIVERY APPARATUS AND METHOD

(71) Applicant: Accelerated Ag Technologies, LLC, Ames, IA (US)

(72) Inventors: Steven Becker, Johnston, IA (US); Jason Cope, Ankeny, IA (US); Wyatt Eugene Fox, Minburn, IA (US); Jared Alan Johnson, Urbandale, IA (US); Jason Kelsick, Elkhart, IA (US); Jacob Thomas Klocke, Polk City, IA (US); Chase Matthew Kramer, Ankeny, IA (US); Zachary Andrew Kreider, Ankeny, IA (US); Brian Glen Walter, Nevada, IA (US); Luke Robert Wille, Holy Cross, IA (US)

(73) Assignee: PowerPollen, Inc., Ames, IA (US)

(21) Appl. No.: 18/606,851

(22) Filed: Mar. 15, 2024

Related U.S. Application Data
(60) Provisional application No. 63/490,611, filed on Mar. 16, 2023.
(60) Provisional application No. 63/524,385, filed on Jun. 30, 2023.

(51) Int. Cl. A01H 1/02

(52) U.S. Cl. CPC A01H 1/027

(57) ABSTRACT

Provided is a precision metering and delivery apparatus and method of use for the precise distribution of viable pollen in diluent or other particulate material via metering and delivery of said pollen or other particulate material, said precision metering apparatus comprising a metering apparatus, a delivery system, and a storage device. Apparatuses of the present invention ensure that the pollen or other particulate material is not harmed during the delivery and that the pollen is applied in the correct density or amount.

### CROSS REFERENCE TO RELATED APPLICATIONS

This application claims priority from U.S. Provisional Application Ser. No. 63/490,611 filed Mar. 16, 2023, and entitled PRECISION METERING DEVICE AND METHOD, as well as U.S. Provisional Application Ser. No. 63/524,385 filed Jun. 30, 2023, and entitled SELF-ADJUSTING APPLICATOR FOR ROW CROPS. The contents of U.S. Provisional Application Ser. Nos. 63/490,611 and 63/524,385 are hereby incorporated in their entirety by reference.

### FIELD OF THE INVENTION

This invention relates generally to precise metering and delivery apparatuses and their use in dispensing and applying particulate substances in agricultural settings, such as traditional fields used for row crops. In particular, this invention discloses a bulk particulate material application system that allows precise metering of particulate material by compressing or dosing said particulate matter and then dispersing it on one or more rows of plants.

### BACKGROUND

The current invention has application to the field of pollination and other crop production practices, including but not limited to seed, grain, vegetable, ornamental, industrial and fruit crop production practices. During the growth period of many crops, it may be beneficial to apply pollen or other particulate matter to said crops. Other particulates are extensively used in agriculture including, but not limited to, particulate formats of fertilizers, fungicides, small seeds and pesticides. Such particulates may be applied to crops on one or more occasions during the growing season. The substances may need to be applied to particular parts of each plant (i.e., flowers, leaves, roots, fruits, etc.), or at a particular height on each plant.

The current invention is particularly suited to the intentional application of pollen, which may be mixed with additional particles which support pollen and plant health. This pollen is applied, for example, to plants that are receptive to the pollen. Said pollen can either be genetically similar to said crops (i.e., self or sib-pollen) and is applied in order to substitute or supplement inadequate natural pollination, or genetically different from said crops (i.e., cross pollination) and is used to effect hybrid crop production or to aid in plant breeding processes.

Intentional pollination is used to produce specific hybrid seeds or plants, to pollinate plants when natural pollination has failed or is particularly poor, when genetic purity is desired, to reduce cost of goods produced, to cross plants which do not typically cross well under natural conditions, to supplement natural pollination, and/or to pollinate plants as needed for any other reason. In such circumstances, pollen must be applied to plants in such a manner to ensure that the pollen reaches the stigma of the receptive flowers in appropriate quantities. The metered application of pollen requires an applicator apparatus that ensures the pollen is not harmed during the delivery, and that the pollen is applied in the correct density or amount.

Pollen can be classified as either orthodox or recalcitrant, based on the water content at the time of dispersal, sensitivity to desiccation, accumulation of biochemical components, and morphology of the pollen grain (Pacini & Dolferus (2016) in Understanding Reproductive Stage Stress Tolerance, Abiotic and Biotic Stress in Plants—Recent Advances and Future Perspectives, ed. A. Shanker (London: Intech), 703-754). Mature pollen grains in orthodox species are typically desiccated at the time of their dispersal and generally have less than 30% water by weight, range in size from 30 to 100 μm, and have one to six furrows and pores on the pollen grain. Examples of orthodox pollen species include Fabaceae species (beans) and most Lamiaceae species (mint, rosemary and other herbs) and many trees, such as maples and oaks. In recalcitrant pollen species, the pollen typically is released with large percentages of water (greater than 30% by weight) and the pollen desiccates on its journey to the female flowers. Recalcitrant pollen is typically 15 to 30 μm or 70 to 150 μm in diameter, has no furrows, and 0-12 (or more) pores on the grain. Examples of recalcitrant pollen species include almost all Poaceae species, squash and pumpkins (Cucurbita pepo), and spinach (Spinacia oleracea) as well as some trees, such as birch (Franchi et al. (2011) J. Exp. Bot. 62:15 5267-5281).

Recalcitrant pollen must be handled very careful because it is much more sensitive to desiccation and other forms of physical damage, and has a very short lifespan. Viability can be lost in minutes to hours depending on species and environmental conditions. Exposure to dry air and high temperature is particularly detrimental to pollen viability and longevity once it is shed from the plant. In particular, pollen from the Poaceae (Gramineae) family of plants, commonly referred to as grasses, is particularly vulnerable and short-lived (Barnabas & Kovacs (1997) In: Pollen Biotechnology For Crop Production And Improvement. (1997). Sawhney, V. K., and K. R. Shivanna (eds). Cambridge University Press. pp. 293-314). This family of plants includes many economically important cereal crops, including maize. Accordingly, there is a need in the industry for metered applicators that do not damage pollen or render it inviable. The applicator of the present invention is specifically designed to handle recalcitrant pollen gently without causing significant damage, thereby maximizing the pollen grain lifespan and pollination potential.

The applicator may be used on any agricultural crop, including seed, grain, vegetable, fruit, tree, and ornamental crops. In particular, the invention is suitable for use on crops that are typically grown in rows. Such plants include, but are not limited to, economically important crops such as soybeans, alfalfa, sunflowers, canola, rice, cotton, hemp, and cereal crops such as corn, wheat, barley, pearl millet, sorghum, and oats.

### BRIEF DESCRIPTION OF THE FIGURES

FIG. 1 is a front elevation view of a first embodiment of the present invention attached to a piece of field equipment.

FIG. 1a is an enlarged view of the embodiment of FIG. 1.

FIG. 2 is a perspective view of a shaftless auger of the present invention.

FIG. 3 is a perspective view of a dosing apparatus of the present invention.

FIG. 4a is a top plan view of a dosing plate of the present invention.

FIG. 4b is a top plan elevation view of another embodiment of a dosing plate of the present invention.

FIG. 5 is a front view of another embodiment of the present invention.

FIG. 6 is a front perspective view of the embodiment of FIG. 4 showing the embodiment in a normal operational configuration.

FIG. 7 is a front perspective view of the embodiment of FIG. 5 showing the embodiment in a travel configuration.

FIG. 8 is a side view of the embodiment of FIG. 5 showing the position of the shaftless auger.

FIG. 9 is another side elevation view of the embodiment of FIG. 8, wherein the shaftless auger is stored vertically for transportation.

FIG. 10 is a cutaway view of the auger chute of the embodiment of FIG. 5.

FIG. 11 is a front perspective view of an applicator of the present invention.

FIG. 12 is a rear perspective view of an applicator of the present invention.

FIG. 13 is a front elevation view of an applicator of the present invention.

FIG. 14 is a front elevation view of a self-adjusting pivot of the present invention.

FIG. 15 is a perspective view of the connection between at least one pivot member and the pivot point of an apparatus of the present invention.

FIG. 16 is a front perspective view of a self-adjusting applicator of the present invention showing the applicator tilting in a first direction.

FIG. 17 is a front perspective view of a self-adjusting applicator of the present invention showing the applicator tilting in a second direction.

FIG. 18 is a front perspective view of a third embodiment of the present invention attached to a piece of field equipment.

FIG. 19 is a side perspective view of a metering apparatus of the embodiment shown in FIG. 18.

FIG. 20 is a top perspective view of the distribution chamber and blower fan of the embodiment shown in FIG. 18.

FIG. 21 is another perspective view of the distribution chamber of the embodiment shown in FIG. 18 with the cover removed.

FIG. 22 is another top perspective view of the distribution chamber of the embodiment shown in FIG. 18.

FIG. 23 is another perspective view of the top of the distribution chamber shown in FIG. 18.

FIG. 24 is a front perspective view of the storage receptacle of the embodiment shown in FIG. 18.

FIG. 25 is a front elevation view of the auger chute of FIG. 10 with particulate matter contained within the auger chute.

FIG. 26 is a perspective view of the dosing apparatus of FIG. 3 showing the compression of particulate matter into a puck.

FIG. 27 is a front perspective view of a distribution chamber of the present invention, shown without a dosing plate to illustrate the extrusion of particulate matter from the compression tube.

FIG. 28 is a front perspective view of the distribution chamber of FIG. 27 with a dosing plate showing the metered shaving of the extruded particulate matter.

FIG. 29 is a front perspective view of the embodiment of FIG. 5 showing the distribution of pollen onto a tall row crop.

FIG. 30 is a side perspective view of the embodiment of FIG. 19 showing the particulate matter chambers loaded with pollen.

FIG. 31 is another side perspective view of the embodiment of FIG. 30 showing the activation of the pistons to dispense pollen from the particulate matter chambers.

FIG. 32 is a top perspective view of the embodiment of FIG. 21 showing the distribution of pollen.

FIG. 33 is a front elevation view of the embodiment of FIG. 17 showing the distribution of pollen onto a row crop.

FIG. 34 is a front elevation view of another embodiment of the invention.

FIG. 35 is a rear elevation view of the embodiment of FIG. 34.

FIG. 36 is a side elevation view of the embodiment of FIG. 34.

### SUMMARY OF THE INVENTION

Provided are embodiments of a precision metering and delivery apparatus for applying particulate material via at least one applicator to a plurality of plants. The precision metering and delivery apparatus may comprise at least one dosing apparatus. This dosing apparatus may comprise a compression device, such as a shaftless auger contained within a compression tube, such as an auger tube, to alter the density of the dispensed material, along with a metering device, such as a rotating dosing plate to precisely meter pollen or other particulate material. The shaftless horizontal auger of the present invention is configured so that the auger's flighting is not coextensive with the end of the auger tube. This allows the buildup of material as it leaves the flighting and is no longer directly moved by the auger. The horizontal auger helps transition the material from a disaggregated, free-flowing powder to a semisolid disk. In other embodiments, the dosing apparatus may comprise a compression tube engaged with a piston to compress the pollen. Such a dosing apparatus may further comprise an impeller to shave and meter the pollen. In still other embodiments, the dosing apparatus may comprise a belt located underneath a storage bin to meter the pollen. The belt may further comprise small scoops to scrape off more pollen and move it to the delivery system. In still further embodiments, the dosing apparatus may comprise a chainmail belt wherein pollen falls into the gaps between the chainmail as it is rotated past the pollen storage bin and is moved to the air delivery system.

The precision metering and delivery apparatus further includes a delivery system, such as an air delivery system. In some embodiments, the precision metering and delivery apparatus further includes at least one storage bin. In some embodiments, the metering device shaves, swipes, or slices the disk of particulate matter by means of a dosing plate located at the end of the compression tube to evenly meter said particulate matter while not degrading pollen viability. The metered particulate matter may then be transported to the desired application location by the air delivery system. In some embodiments, the air delivery system may split to feed one or more applicator devices. In some embodiments, the storage bin feeds pollen to the auger in order to prepare the pollen to be metered and distributed.

In a first embodiment of the invention, a large particulate metering system is provided comprising a storage bin, a metering system, a metering device, a compression device, and an air delivery system. The bin dispenses particulate material into the compression device, which compresses the particulate material into a cylindrical disk or puck. The cylindrical disk is then shaved by the metering device, which shaves the cylindrical disk at a rate dictated by the metering system. This rate may vary based on the size of the delivery system, by a rate of application based on area, by the speed of travel calculated from GPS feedback combined with an application rate based on area, or any combination thereof. The shaved pollen or other particulate material is then delivered to crops by the air delivery system through one or more applicators.

Embodiments of the invention may be adapted for use in one or more of a field, greenhouse, vertical farming facility, hoop house, and a high tunnel. The plants may be in rows, blocks, or other configurations that permit the usage of the applicator in the vicinity of the plants. Further embodiments of the invention may be adapted for operation by means of, but not limited to, manpower, all-terrain vehicle, tractor, a robotic applicator, aerial or drone based, motor vehicle or a stationary applicator.

### DETAILED DESCRIPTION

Example embodiments will now be described more fully with reference to the accompanying drawings. Example embodiments are provided so that this disclosure will fully convey the scope of the mechanism and operation to one having at least ordinary skill in the art. Numerous specific details are set forth such as examples of specific components, devices, and methods, to provide a thorough understanding of embodiments of the present disclosure. It will be apparent to one having at least ordinary skill in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers and/or sections should not be limited by these terms. These terms may be only used to distinguish one element, component, region, layer or section from another element, component, region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Disclosed is a unique large volume precision metering apparatus for use in agricultural applications, including, but not limited to, for the application of pollen to row crops. The disclosed applicator has the ability to accurately meter and deliver pollen or other particulate matter using a simple, scalable method. Such technology and methods may be used in association with any plants for which it is desired to deliver or apply solid particulate material. Such material may be applied directly to a plant, to the ground, or both. For ease of discussion and understanding, the following detailed description may refer to the invention for use with dispersing pollen. However, it should be appreciated that the technology and methods may be used with any solid particulate material and any plants. The invention has particular applicability towards compositions of particulate material with variable moisture contents, such as pollen, which have traditionally been difficult to precisely meter and distribute due to the variable flow characteristics of the particulate matter. Pollen specifically has the tendency to conglomerate when stored which dramatically inhibits its flowability and pollen viability.

As used in this disclosure, “pollen” may refer to either pure pollen or it can refer to pollen that has been mixed with other additives. Furthermore, the pollen may be freshly-collected pollen, recently-collected pollen, or pollen that was previously collected and subjected to short-term or long-term storage, including pollen that has been maintained at cool or cold temperatures, including cryopreserved pollen. When additives are combined with the pollen, they may be mixed with the pollen at specified additive to pollen ratios. The additives may be non-living particles, living particles, or they may be a combination of living and non-living particles. The additives may include, for example, diluents, anti-flocculants, absorbent materials, materials intended to separate particles, or be materials with other uses such as, but not limited to, additives that help maintain the viability of the pollen or other living particles.

For the purposes of this disclosure, the term “viable” or “viability” is used to describe pollen that is able to germinate and grow a pollen tube to at least a length twice the diameter of the pollen grain. In addition, pollen can be judged viable by demonstration that the cellular nature of the material remains integral and is judged to maintain intactness such that normal cellular processes of metabolism and intracellular functioning is possible. The viability of pollen can be assessed in numerous ways, including, but not limited to, assessment of pollen tube growth on artificial media or excised stigmas or styles, assessment of cellular intactness by vital staining of numerous sorts, absence of electrolyte (e.g., potassium) leakage, impedance flow cytometry, and seed set. Viable pollen can successfully germinate and commonly possesses the vigor necessary to promote fertilization and initiation of seed development. Not all viable pollen is also fertile pollen. In some cases, even when a pollen grain is viable and commences with pollen tube growth, it may lack the vigor necessary to reach the ovule and promote fertilization. Non-viable pollen grains cannot successfully germinate. Viability can refer to a single pollen grain or a population of pollen grains. When a percentage value is used to describe pollen viability, the value is typically being applied to a population of pollen.

It is important to prevent physical stress to pollen and other living particulates during handling, particularly when recalcitrant pollen is being used, or when using other living particulates, the viability of which is impacted by stressors such as heat, humidity, friction, crowding, compression, or other stressors. In particular, handling of the pollen by means of vibration, forced air, rotation, physical force or other means can be disruptive to pollen membrane integrity, at least in part as a result of the friction experienced during the distribution process. Accordingly, embodiments of the present invention seek to minimize the stress applied to pollen, other living particulates, or other non-living particulate matter being distributed in a growing environment or applied to a crop. In some cases, additives may be used to reduce the impact of stressors. For example, additives may be used to reduce the effects of high humidity, thereby reducing clumping or aggregation of living or non-living particles. Research has demonstrated that live pollen grains that come into contact with the contents of burst dead pollen cells are adversely affected by such contact, reducing viability and killing the pollen cells (U.S. patent application Ser. No. 16/028,626). As such, pollen particles may be blended with additives to reduce contact with dead pollen contents. Other living particles that can be applied using the invention may include actual insects, such as those used in biological control regimes to combat crop pests or plant diseases, which may be gently blended with additives to improve their viability, prevent damage to the insects, to absorb waste products, and to provide nutrition during storage. Other living particles that can be applied using the invention may include bacteria, fungi, or virus particles that are being applied to treat crop pests or plant diseases. Bacteria and fungi, for example, may be blended with an additive that includes supporting nutrients for growth, that reduces damage, or otherwise protects the particles or improves their viability. In all cases, both living and non-living particles of all kinds may also be blended with additives that serve as diluents to improve the efficacy of delivering a metered dose to the crop.

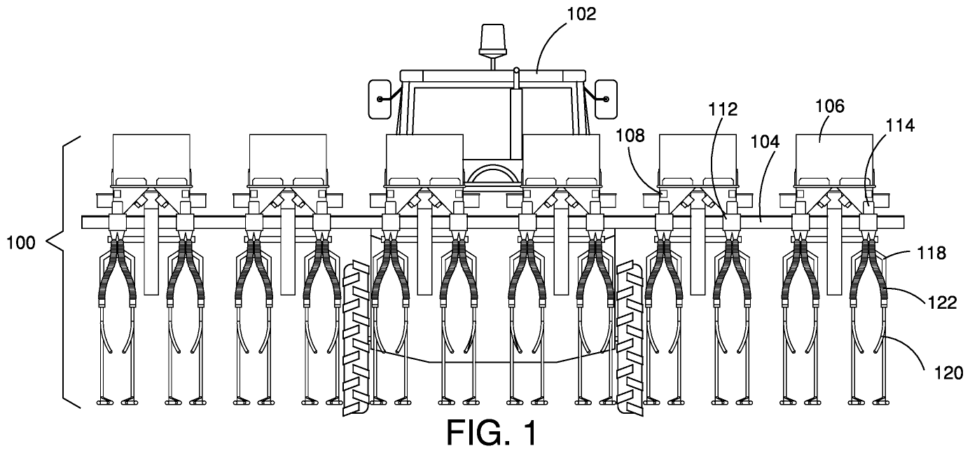

In a first embodiment of the invention, a precision metering and delivery apparatus is provided, as shown in FIG. 1. In the illustrated example, the precision metering and delivery apparatus is adapted for attachment to field machinery, such as a tractor. However, the precision metering and delivery apparatus may be adapted for any method of moving plants through the apparatus, including but not limited to manpower, all-terrain vehicle (ATV), robotic applicator, other field machinery, aerial machinery, motor vehicle, or a stationary applicator wherein plants or portions thereof are moved through the applicator by a conveyor or other mechanism. In one embodiment, the precision metering and delivery apparatus may be attached to a vehicle driven by an operator. In another embodiment, the precision metering and delivery apparatus may be attached to an autonomous vehicle, such as a self-driving car, a self-driving tractor, or a self-piloting drone.

The precision metering and delivery apparatus may include at least one storage device or hopper. In some embodiments of the invention, the storage device may be a storage bin. In one embodiment, the storage bin is removably attached to an auger chute, such that the storage bin may be removed from the auger chute in order to enable ease of transport, or to clean accumulated material from the walls of the storage bin. In one embodiment, the storage bin is removably attached with a set of toggle clasp latches to enable toolless removal of the storage bin. However, other means of removably attaching the storage bin may be used, such as screws or other fasteners, without departing from the scope of the invention. The storage bin may alternatively be permanently fixed to the auger chute using permanent fastening means, such as by welding, or any other suitable method that would provide a sturdy connection, without departing from the scope of the invention. In one embodiment of the invention, the storage bin is rectangular in shape and is positioned on top of two equally sized auger chutes. These auger chutes consist of a larger opening end and a smaller compression tube feeding end formed by three vertical sides and one sloped side. In the preferred embodiment, the sloped side is configured to be at a slope between 15 and 75 degrees, preferably between 30 to 60 degrees, most preferably between 40 and 50 degrees.

In one embodiment, the auger chutes are configured such that the sloped side of the two auger chutes meet at the top of the sloped side of each chute, such that material deposited into the auger chutes travels down the sloped side of each chute away from the opposing chute. In another embodiment, the auger chutes further include one or more agitators located along the sloped or vertical side of the auger chute. In the one embodiment, the agitators are made up of a series of horizontal bars connected by a vertical bar that corresponds to the length of the sloped side of the auger chute and continues above the material exit, along with a motor system attached to the vertical bar. When in use, a motor system attached to the vertical bar will rapidly move the ends of the horizontal bars up and down. This movement allows the horizontal bars to remove stuck pollen from the sloped side of the auger chute, which prevents accumulation of pollen in the auger chute. Agitators of the present invention could also take the form of a thumper, a pneumatic piston, a stir agitator, a ribbon blender, or combinations thereof.

Another issue that can occur in pollen distribution is bridging. Bridging is a self-created arch of pollen or other particulate material that can form above the outlet of a hopper as it empties. Bridging occurs when the ends of the arch are held up by wall friction. The agitators of the present invention combat bridging by breaking up accumulated pollen in the auger chute before an arch can form. The bin may also be pressurized to allow for a more uniform flow of material.

The invention may comprise a compression device to compress pollen or other particulate matter in order to enable consistent metering of said pollen or other particulate matter. In one embodiment, the compression device is a shaftless auger contained within a compression tube and attached to the bottom of the auger chute. In another embodiment, the compression device may be a piston contained within a compression tube and attached to the bottom of a distribution chamber.

In one embodiment of the invention, the shaftless auger is positioned such that the flighting of the auger is not coextensive with the auger tube. The auger tube extends past the flighting of the auger, providing a compression zone to collect the pollen/diluent or other particulate substances. In the preferred embodiment, the auger includes a mechanism to adjust the location of the auger within the auger tube to increase or decrease the size of the compression zone. In one embodiment, this compression zone varies between two inches to four inches in length at the end of the auger tube, depending on the desired level of pollen cohesiveness leaving the auger. The compression zone will preferably be between two inches to four inches, such as between three inches to four inches, most preferably between three to three and a half inches in length. The ideal compression zone length depends on the material properties of the substance being compressed, as well as the length and cross-sectional area of the auger tube. Smaller tubes and augers would allow for higher precision at lower volumes. Larger tubes and augers would allow for higher application rates and a large number of rows to be fed from a single unit. Additionally, smaller tubes and augers would require smaller compression zones to reach desired levels of particulate aggregation, whereas larger tubes and augers require larger compression zones. Substances that have larger moisture contents tend to pack together more easily, so shorter compression zones may be more desirable. Substances with lower moisture contents tend to be more free-flowing and may require larger compression zones to form a uniform extrusion surface. Generally, larger compression zones will produce firmer discs of pollen, whereas a shorter compression zone will produce less cohesive disks. The ability to sense puck travel and/or density will be used to provide an automated feedback loop to ensure rate stays consistent. After leaving the compression zone, the pollen is exposed to a cutting device capable of breaking up the extruded disk in a predominantly uniform fashion at a metered rate. In one embodiment of the invention, the metering device consists of a dosing plate with a center of rotation at the center of the auger tube connected to a rotating mount. In the preferred embodiment, the dosing plate consists of a disk having a circular opening. The circular opening is sized to correspond with the exit of the compression tube. The circular opening is intersected by one or more dividing bars, wedges, wires, impellor, fan, perforated metal, grates, chainmail, or other metering devices that shave the pollen from the extruded disk as they rotate across the surface of the extruded disk. This rotating dosing plate shaves off all the pollen or other material that has been displaced from the end of the auger tube. The rotating dosing plate of the preferred embodiment enables more reliable metering of the pollen when tied to speed than would be provided by a gravity feed, wherein the pollen would be extruded from the shaftless auger, flighted auger, piston, chain or cable conveyance, or belting conveyance until the force of gravity acting on the extruded pollen would cause it to detach and fall. Though the metering device of the preferred embodiment has been described above as a rotating dosing plate, other types of metering devices, such as a squirrel cage meter or rotating impellor in axial or perpendicular orientations, may be used without departing from the scope of the invention. A squirrel cage meter comprises a rotating impeller cage positioned at the end of the compression tube. The cage is positioned so that the impeller blades cut across the end of the compression tube when the cage is rotated, shaving off extruded pollen or other material.

Connected to the shaftless auger and metering device is the air delivery system of the present invention. The air delivery system transports the metered pollen or other materials to the desired location for dispersal on a plant or a row of plants. In the preferred embodiment, a single unit of the precision metering and delivery apparatus will have at least one distribution chamber that is connected to the metering device. In some embodiments of the invention, the distribution chamber is rectangular or circular in shape, with a rectangular or circular horizontal cross section. The distribution chamber has a top opening to accommodate a blower fan located above the distribution chamber. The blower fan may be located directly above the distribution chamber and connected to the distribution chamber. The blower fan may be connected to the distribution system by a system of blower tubes, which allow the blower fan to be positioned further away from the distribution chamber. Preferably, the blower fan is not directly connected to the distribution chamber. At least a small amount of spacing is preferable to allow the output of the blower fan to reach a laminar flow, which aids even distribution of material. This amount of spacing can be small, such as an inch, or it can be larger, such as 3 to 4 feet. Additionally, in some embodiments, the bins and tubes could be placed under pressure, such as positive pressure, in order to aid distribution of particulate material or to clean out the system.

To this end, the blower fan is connected to the distribution chamber either by flexible tubing or a stiff connection. In the preferred embodiment each metering device will be connected to a distribution chamber. In some embodiments of the invention, the distribution chamber is divided in half by an adjustable divider. Each half of the chamber covers half of the output area of the auger tube and metering device such that half of the overall extruded and shaved pollen will enter each side of the distribution chamber. At the bottom of the distribution chamber is one or more sloped distribution funnels wherein the output located at the bottom of the funnel is connected to a pollen applicator. In some embodiments, each distribution chamber has two funnels corresponding to the two halves of the distribution chamber. The distribution funnel is connected to the pollen applicator by a corrugated tube. In the preferred embodiment, the interior of the corrugated tube has a smooth bore to prevent the buildup of particulate matter in the spaces between the corrugation. In some embodiments, the delivery system further comprises at least one applicator to dispense particulate material. In some embodiments, the applicator may be a self-adjusting row applicator with at least two applicator tips, where the applicator is configured to receive at least one plant, such as a plurality of plants in succession as the applicator moves through one or more rows of plants. In one embodiment, the self-adjusting applicator comprises a single unit or “head” configured to receive a row of plants. The preferred embodiment of the invention contains a plurality of heads such that pollen can be applied to multiple rows of crops simultaneously through the plurality of heads. The self-adjusting applicator may comprise at least one guide member configured to successively sense each of the plurality of plants in the row. If a plant is located a different lateral distance from the longitudinal center than the immediate previously sensed plant, the guide member actuates the self-adjusting applicator to move the at least one applicator.

In another embodiment of the invention, the compression device is a vertical piston contained within a storage receptacle. The compression device feeds compressed pollen to a metering device, which may comprise a vertical impeller, by pushing the pollen contained within the storage receptacle into the metering device using the vertical piston. In one embodiment of the invention, the distribution chamber comprises an impeller housing and impeller housing cover. This embodiment of the invention further comprises an air delivery system, which comprises one or more fans attached to the top of the distribution chamber. The vertical impeller and air delivery system combine to move pollen shaved by the vertical impeller to the material outlet. The material outlet is connected to a pollen applicator, such as a self-adjusting applicator, by a corrugated tube.

In some embodiments, the self-adjusting applicator further comprises a self-adjusting pivot, which may include a pivot point, a biasing member, at least one pivot member, and at least one guide member. The biasing member may bias the self-centering applicator in a neutral or balanced position. The neutral or balanced position may be at the longitudinal center of the row. The at least one guide member may “sense” the plants by touching or otherwise coming into contact with the plants. This contact may cause the guide member to move in a lateral position, such as following an arcuate path outward and upward from the neutral or balanced position. The movement of the guide member may overcome the bias of the biasing member. This may cause the pivot member to pivot about the pivot point. In some embodiments, the self-adjusting applicator may include two pivot members.

The self-adjusting applicator may include at least one applicator tip, and the pivot member may be engaged with the applicator such that movement of the pivot member may move the at least one applicator. In some embodiments, the applicator tip may be a nozzle or an atomizer. The self-adjusting applicator is preferably configured to apply pollen or other particulate matter. However, it is foreseeable that one having at least ordinary skill in the art could modify the self-adjusting applicator to dispense other materials, such as other living particulate materials such as insects. The self-adjusting applicator may include at least one height adjustment member configured to adjust the height of the at least one guide member with respect to the ground.

Moreover, in one embodiment, pollen applicator tips are located on each side of said pollen applicator, such that pollen is applied to both sides of a row of plants being fed through said applicator. In one embodiment of the present invention, the applicator device has two applicator tips, where each applicator tip is being fed by a corrugated tube. However, it is foreseeable that one having at least ordinary skill in the art could modify the air delivery system by adding or removing tubes, such that the air delivery system consists of a single tube branching to feed into both sides of a single applicator, a plurality of tubes feeding into a plurality of applicators, a single tube feeding into a plurality of applicators, a plurality of tubes feeding into a single applicator, or any number of permutations thereof without departing from the scope of the invention. Accordingly, embodiments of the invention may have a plurality of tubes connected to a single metering device, such that a plurality of rows would be fed by a single metering device. It is conceivable that a single metering device could feed multiple rows, such as two rows, three rows, four row, five rows, six rows, or more.

In an example embodiment practicing the claimed invention, the precision metering and delivery apparatuses are attached to the field machinery via one or more supports, such as a support network, which one having at least ordinary skill in the art may modify based on factors including, but not limited to, number of the precision metering and delivery apparatuses, type of field machinery or other propelling force, and type of plant. This support network provides a framework to which the precision metering and delivery apparatuses may be attached.

Shown in FIG. 1 is a mounted precision metering and delivery apparatus 100 of the present invention. In the embodiment shown in FIG. 1, a plurality of precision metering and delivery apparatuses 100 are shown connected to a piece of field machinery 102 by a support network 104. Also shown is a storage bin 106 for the retention of pollen. The storage bin 106 is shown positioned above an auger chute 108 of the present invention. Below the auger chute 108 is a compression tube 109 of the present invention (not shown). In the described embodiment, the compression tube 109 is an auger tube 110 which compresses the fed pollen or other particulate material into a semi-solid puck 111 (not shown). While in use, the auger tube 110 is maintained in a predominately horizontal position with respect to the ground. Shown also in FIG. 1 is a distribution chamber 112 of the present invention, located at the end of the auger tube 110. The distribution chamber 112 is shown connected to a blower fan 114. Also shown in FIG. 1 are adjustable row arch applicators 118. The adjustable row arch applicators 118 comprise sets of applicator tips 120 which are connected to the distribution chamber 112 by sets of distribution tubes 122.

Shown in FIG. 1a is a closer view of a mounted precision metering and delivery apparatus 100 of the present invention. The precision metering and delivery apparatus 100 is shown connected to a piece of field machinery 102 by a support network 104. Also shown is a storage bin 106 for the retention of pollen. The storage bin 106 is shown positioned above an auger chute 108 of the present invention. Shown also in FIG. 1a is a distribution chamber 112 of the present invention, located at the end of the auger tube 110 (not shown). The distribution chamber 112 is shown connected to a blower fan 114. In the embodiment shown in FIG. 1a, the blower fan 114 to the distribution chamber 112. Also shown in FIG. 1 are adjustable row arch applicators 118. The adjustable row arch applicators 118 comprise sets of applicator tips 120 which are connected to the distribution chamber 112 by sets of distribution tubes 122.

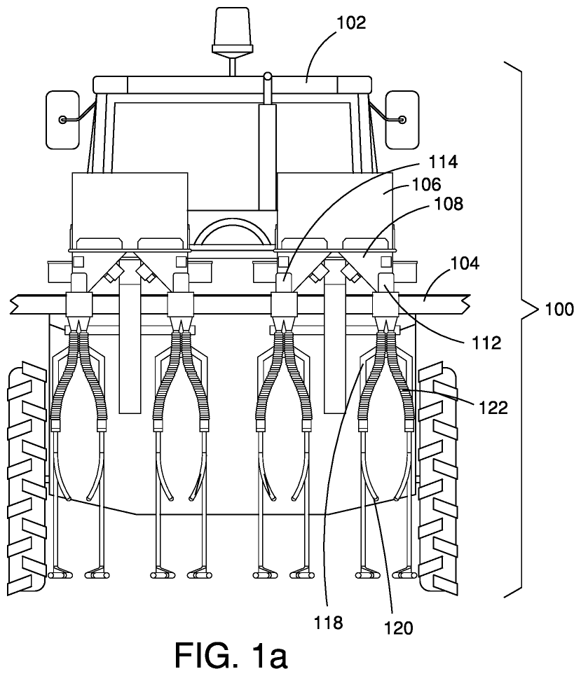

Shown in FIG. 2 is an auger assembly of the present invention. The auger tube 110 is shown containing a shaftless auger 124 of the present invention. The shaftless auger 124 shown comprises an auger motor 126 connected to the stem and flighting 128 of the auger. The auger tube 110 shown in FIG. 2 further includes a rectangular opening 130 where pollen can be fed from the storage bin 106 (not shown) to the shaftless auger 124 through the auger chute 108 (not shown). In the shown embodiment, the rectangular opening 130 includes a plurality of connection holes 132 for the insertion of screws or other fastening means in order to connect the auger chute 108 to the shaftless auger 124. However, other means of attachment, such as a permanent attachment means such as welding, or other types of tooled and tool-less attachment may be used to connect those components without departing from the scope of the invention. Also shown in FIG. 2 is a compression zone 134 of the present invention. As discussed above, the compression zone 134 comprises a section of the auger tube 110 between the end of the flighting 128 and the terminal end of the auger tube 110.

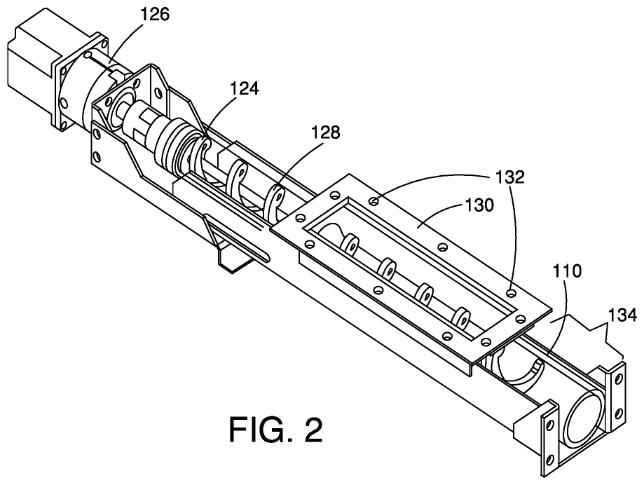

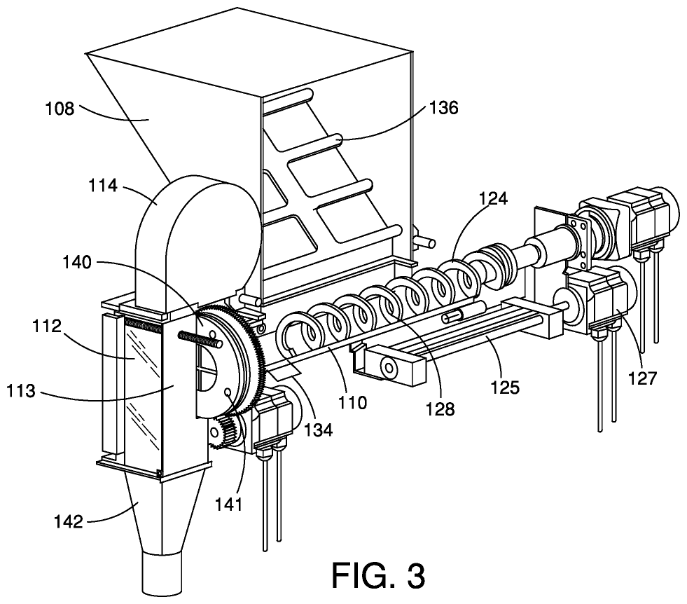

In FIG. 3, a cutaway view of dosing apparatus 107 of the present invention is shown. The auger chute 108 is shown with an agitator 136 for the dislodging of stuck pollen or other material from the auger chute 108. Also shown is the shaftless auger 124. The shaftless auger 124 is shown with a cut away view of the auger tube 110 to better show the flighting 128 of the auger. The auger 124 includes an auger position adjustment mechanism 125 which is capable of adjusting the position of the auger 124 laterally within the auger tube 110 to either increase or decrease the size of the compression zone 134. In some embodiments, the auger position adjustment mechanism 125 is capable of automatic adjustment of auger 124 position based on sensor feedback to determine whether or not a puck has formed and if said puck is progressing through the auger tube at the correct speed. Below the auger tube 110 is shown an auger position adjustment motor 127 that operates the auger position adjustment mechanism 125 to increase or decrease the compression zone 134 by adjusting the position of the shaftless auger 124 within the auger tube 110. In the shown embodiment, the auger position adjustment mechanism 125 is a screw adjuster connected to a motor 127, where the rotation of the screw by the motor 127 will move the auger 124 along the screw either forwards or backwards. At the end of the auger tube 110 a dosing plate 140 is shown which shaves extruded pollen or other particulate material off of the puck 111 (not shown) formed in the compression zone 134. Due to the cut away view, the embodiment shown in FIG. 3 shows half of the distribution chamber 112 located at the end of the auger tube 110 to better show the dosing plate 140. In the preferred embodiment the dosing plate 140 is attached to a rotation mechanism with a plurality of magnetic studs 141. These magnetic studs 141 facilitate the toolless interchange of different dosing plates 140. In the example embodiment, the dosing plate 140 is configured to maximize the exposed area of the circular opening by having thin dividing bars to shave the pollen or other particulate matter. However, dosing plates 140 having other faces may also be used, as shown in FIGS. 4a and 4b. For example, it may be beneficial in the operation of an apparatus of the present invention to use a dosing plate 140 having a smaller opening when beginning operation of the apparatus to provide more resistance to material leaving the compression tube 109. This smaller opening would help the particulate material in the compression zone 134 to achieve an even surface for the dosing plate 140 to shave off by reducing the flow of pollen from the compression tube. After an even surface is achieved, the smaller opening area dosing plate 140 could be easily switched out for the normal dosing plate 140 for normal operation.

Also shown is a distribution chamber divider 113 comprised of a vertical wall separating the distribution chamber 112 in half, so that half of the shaved pollen would be dispensed into each side of the distribution chamber 112. The distribution chamber divider preferably includes an adjustment mechanism, such as a screw adjuster, which can move the top portion of the divider to the right or left. The adjustment mechanism enables the alteration of pollen distribution such that the sides of the distribution chamber could be altered to receive a larger percentage of shaved pollen. In the embodiment shown in FIG. 3, the distribution chamber 112 is shown with the blower fan 114 connected to the top of the distribution chamber 112. Below the distribution chamber 112 is the distribution funnel 142 that feeds material to an applicator, such as an adjustable row arch applicator 118 (not shown).

Shown in FIGS. 4a and 4b are two different embodiments of a dosing plate 140 of the present invention. FIG. 4a shows a dosing plate 140 having four equally sized openings 143. The dosing plate 140 is configured to have a larger opening area to provide less resistance to extruded pollen or other particulate material. FIG. 4b shows a dosing plate 140 having two equally sized openings 143. The dosing plate 140 is configured to have a smaller opening area to provide more resistance to extruded pollen or other particulate material. While the dosing plate 140 has been described with respect to the pictured embodiments, numerous other sizes and shapes of openings 143 could be used without departing from the scope of the invention. While the openings 143 have been shown as a plurality of circular quadrants, any opening shape may be used without departing from the scope of the invention.

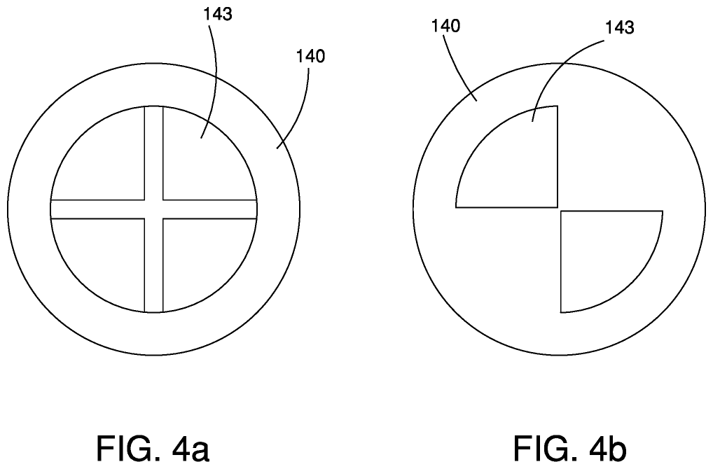

Shown in FIG. 5 is a front view of another embodiment of the present invention showing a metering unit of the present invention configured to distribute particulate matter to two crop rows. The storage bin 106 is shown connected to two auger chutes 108. On the right-side auger chute 108, the distribution chamber 112 is shown in a transparent view to allow the viewing of the dosing plate 140. However, in some embodiments, the front of the distribution chamber 112 may be made of a transparent material, such as glass or plastic, to allow operators to more easily view the extrusion of pollen or other particulate material 196 to ensure proper operation of the auger 124. Above the distribution chamber 112 of the embodiment is a blower fan 114. Below the distribution chamber 112 are shown the distribution funnels 142. The distribution funnels 142 are shown connected to the applicator tips 120 of the adjustable row arch applicators 118 by flexible distribution tubes 122.

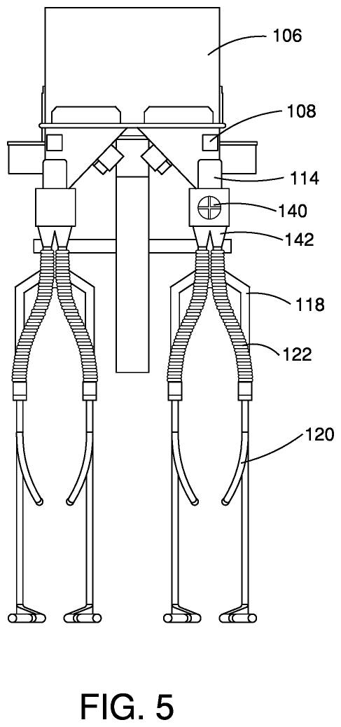

Shown in FIG. 6 is a front perspective view of the embodiment of FIG. 5 The storage bin 106 is shown connected to two auger chutes 108. The auger chutes 108 are shown connected to the support network 104. Above the distribution chamber 112 of the embodiment is a blower fan 114. Below the distribution chamber 112 are shown the distribution funnels 142. The distribution funnels 142 are shown connected to the applicator tips 120 of the adjustable row arch applicators 118 by flexible distribution tubes 122.

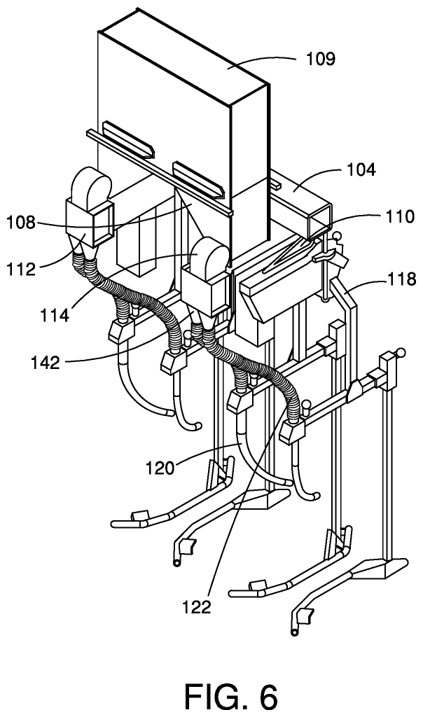

Shown in FIG. 7 is a front perspective view of the embodiment in FIG. 6, wherein the position of some of the components, such as the shaftless auger 124 and auger tube 110 are moved into a travel configuration. When the invention is used in territories having vehicle width requirements, the travel configuration is beneficial to enable tractors or other field machinery 102 having embodiments of the present invention mounted thereon to travel via public roads to reach different application locations without needing to be transported by other equipment. In such a configuration, the auger tube 110 is shifted up, and the distribution tubes 122 would be removed and stored.

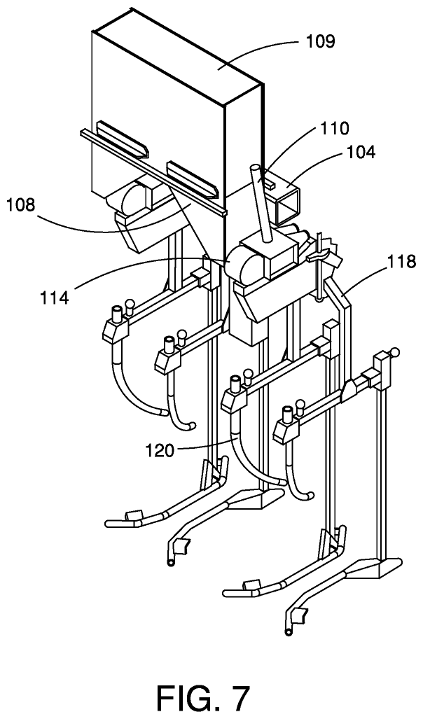

Shown in FIG. 8 is a side view of the embodiment shown in FIG. 5. The storage bin 106 is shown connected to two auger chutes 108. The auger chutes 108 are shown connected to the frame members 105 of the support network 104 Above the distribution chamber 112 of the embodiment is a blower fan 114. To the right of the distribution chambers 112 of the embodiment is the auger tube 110 containing the horizontal shaftless auger 124. Below the distribution chambers 112 are shown the distribution funnels 142. The distribution funnels 142 are shown connected to the applicator tips 120 of the adjustable row arch applicators 118 by flexible distribution tubes 122.

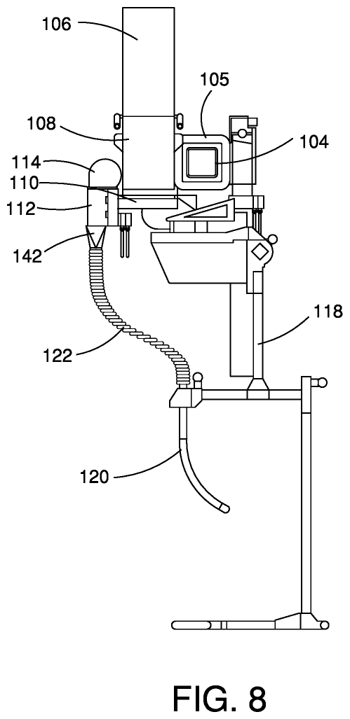

Shown in FIG. 9 is another view of the embodiment shown in FIG. 8. In the embodiment the shaftless auger 124 has been shifted to a vertical orientation for transport. The storage bin 106 is shown connected to the auger chute 108. The auger chute 108 is shown connected to the frame members 105 of the support network 104 To the left of the distribution chamber 112 of the embodiment is a blower fan 114. Above the distribution chamber 112 of the embodiment is the auger tube 110 containing the shaftless auger 124. To the right of the distribution chamber 112 is shown one of the distribution funnels 142 along with applicator tips 120 of the adjustable row arch applicators 118.

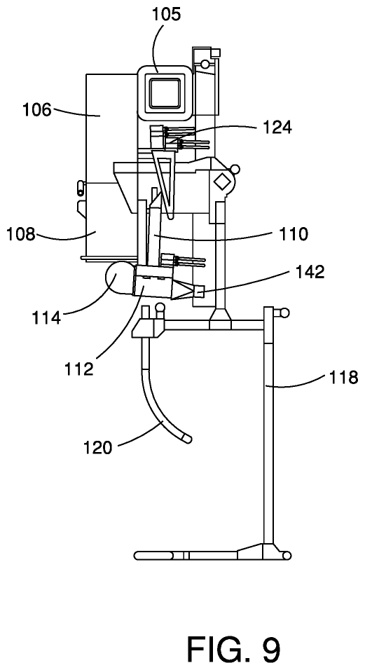

Turning to FIG. 10, shown is a cutaway view of the auger chute 108 of the embodiment of FIG. 3. The position of the agitator 136 is shown. In the shown embodiment, the agitator 136 runs parallel to the slope of the angled wall of the auger chute 108. At the end of the auger chute 108 where the auger chute 108 is connected to the compression tube 109, the agitator 136 continues and angles toward the compression tube 109. Also shown is the agitator motor 138 located on the underside of the sloped side of the auger chute 108.

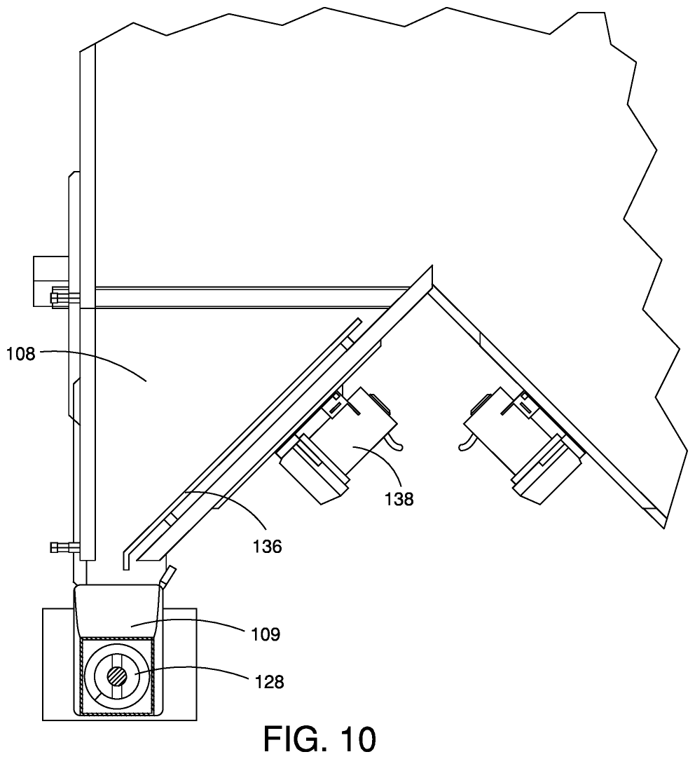

Turning to FIG. 11, a view of a head 142 of an adjustable row arch applicator 118 is shown. The head includes a bracket 144 which connects the head 142 to the support network 104 (not shown). Attached to the bracket 144 is one or more pivot members 146. In the illustrated embodiment, each of said pivot members 146 is attached to one or more braces 148 at the end opposite the bracket 144. The illustrated embodiment includes one brace 148 per pivot member 146. Moreover, the illustrated brace 148 can be described as a generally horizontal bar. However, one having at least ordinary skill in the art will recognize that any size, shape, or type of brace may be used. In addition, the brace 148 is optional, as the components held or supported by the brace 148 could be held or supported directly by the one or more pivot members 146. The braces 148 each hold one or more adjustable length leg members 150. Attached to the adjustable length leg members 150 is at least one guide member 152. In the illustrated embodiment, a pair of guide members 152 extend both forward and rearward (referring to the direction of forward travel) to guide the pivoting members 146 towards a plant or plant row. The guide members 152 are configured to accept one or more plants. In the illustrated embodiments, a set of guide members 152 accepts a series of plants in succession that are planted in a row. Accordingly, the guide bars 152 guide the pivoting members 146 towards the plants as the plants pass between the guide bars 152. In the illustrated embodiment, one or more applicator tips 120 are shown attached to the brace 148 at the opposite end from the adjustable length leg members 150, although any configuration may be used without departing from the scope of the invention.

The illustrated embodiment also includes a self-adjusting pivot 154. As will be described in detail below, the self-adjusting pivot 154 enables the pivot members 146 to pivot with respect to an applicator pivot point 156. In cases where the row spacing is variable, the self-adjusting pivot 154 described herein allows the head 142, including components such as the pivot members 146 and the applicator tips 120 to move into a position that accommodates the variability. Additionally, the self-adjusting pivot 154 accommodates rows with curves or other variations within the planting row. This ensures that the applicator tips 120 remain properly positioned relative to the plant regardless of any variability in the row.

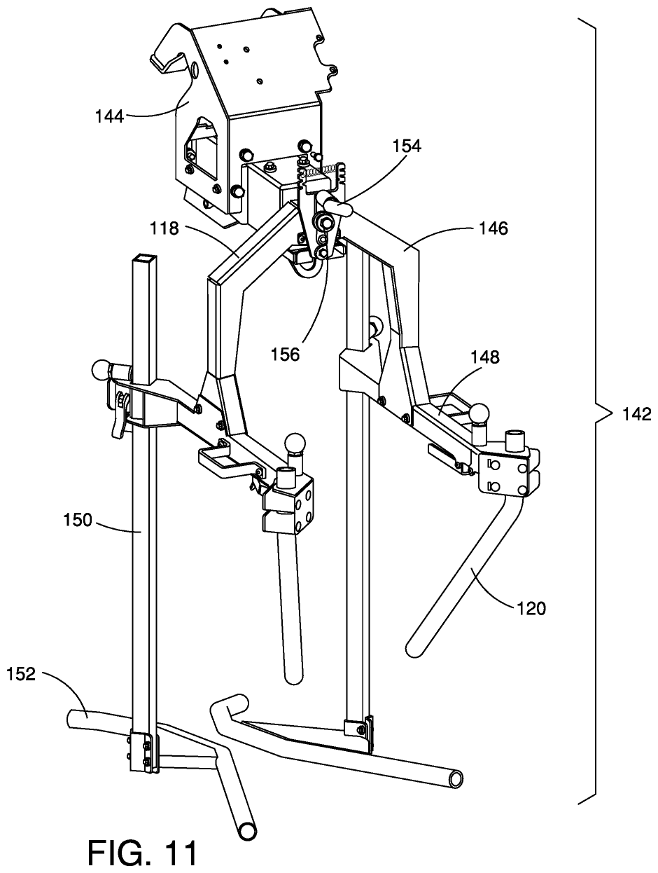

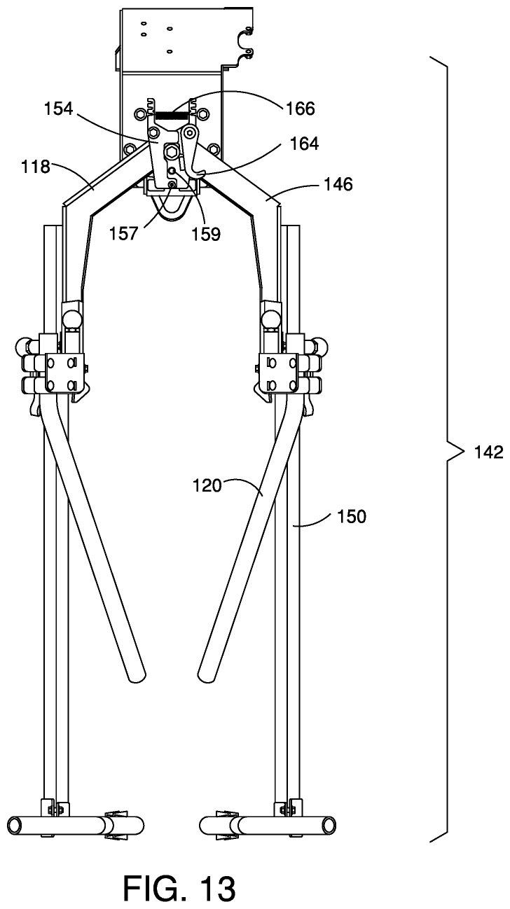

Shown in FIG. 12 is a rear perspective view of a head 142 of an applicator device of the present invention. A rear view of the bracket 144 is shown. In some embodiments, the bracket 144 is configured to attach the head 142 to the support network 104 (not shown). In other embodiments, the self-adjusting pivot 154 may be attached to a support network directly without the use of a bracket. The head 142 includes a self-adjusting pivot 154 attached to the bracket 144 (shown in more detail in FIGS. 14-16). In the shown embodiment, the self-adjusting pivot may be secured to said support network 104 using a connection such as bolts 158. As will be understood by one having at least ordinary skill in the art, any type of connection may be used. The head 142 further comprises pivot members 146 connected to the bracket 144 by the self-adjusting pivot 154, as described in further detail below. The head 142 may further comprise adjustable length leg members 150. In the illustrated embodiment, the length of the adjustable length leg members 150 is adjusted using a pin adjuster 160 in combination with a plurality of equally spaced holes 162 along the length of the adjustable length leg members 150, the equally spaced holes 162 being sized to receive the pin of the pin adjuster 160. However, in other embodiments, members that are not capable of length adjustment may be included.

The adjustable length leg members 150 may have a first and a second end, said first end being closer to said bracket 144 and said second end being further from said bracket 144. Said first end may also be referred to as the proximal end and said second end may also be referred to as the distal end. Although the leg adjustment mechanism has been described as a pin adjuster 160 being used with a plurality of equally spaced holes 162, one having at least ordinary skill in the art will be able to substitute this adjustment method with any number of alternative mechanisms without departing from the scope of the invention. The head 142 may further comprise applicator tips 120 attached to the pivot members 146. The head 142 may further comprise guide members 152 fixed to the distal end of the adjustable length leg members 150. In the illustrated embodiment, the guide members 152 are shaped in a manner so that the opening between the guide bars 152 is broader at the front so as to find plants that are far from center.

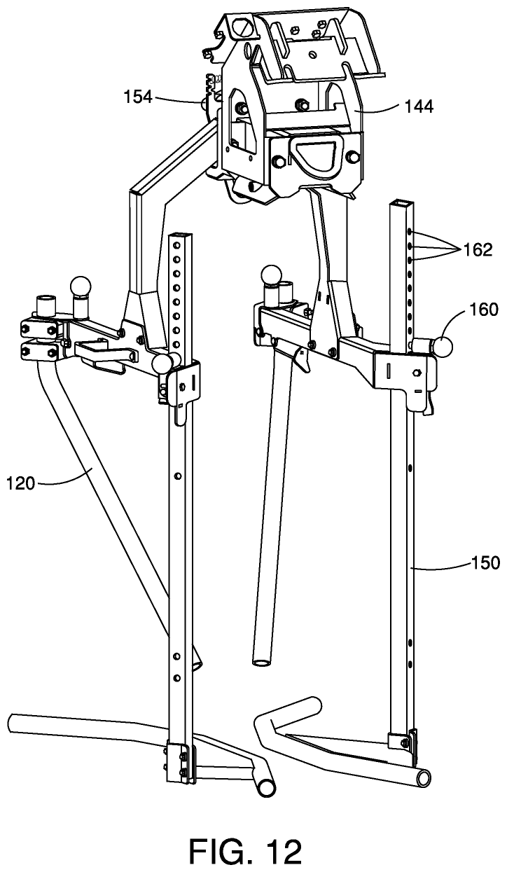

Shown in FIG. 13 is a head 142 of an adjustable row arch applicator 118 of one embodiment of the present invention showing a locking member 164. The head 142 in the illustrated embodiment includes a bracket 144 attached to a self-adjusting pivot 154. The self-adjusting pivot 154 may further include at least one locking member 164. In some embodiments, the locking member 164 may be in the form of an immobilizing latch. The self-adjusting pivot 154 is actuated by an adjusting mechanism 163. In the illustrated embodiment, the adjusting mechanism 163 is a biasing member 165, such as a spring 166. The head 142 further comprises one or more pivot members 146 connected to the self-adjusting pivot 154. In the illustrated embodiment, adjustable length leg members 150 and applicator tips 120 are also included. The adjustable length leg members 150 may be adjusted by a pin adjuster 160. The adjustable length leg members 150 may connect to one or more guide members 152 attached to the lower portion of the adjustable length leg members 150. Also shown is a biasing prong pivot point 157 below the applicator pivot point 156, and an actuating pin 159.

As discussed above, shown in FIG. 14 is an embodiment of a self-adjusting pivot 154 of one embodiment of the present invention. This illustrated design includes a biasing member 165, for example a spring 166. The illustrated embodiment does not require electrical, hydraulic, or pneumatic components, although such components could be included without departing from the scope of the invention. This results in an efficient design that is less vulnerable to component failure. The self-adjusting pivot shown further provides an applicator pivot point 156 about which the pivot members 146 can pivot. The illustrated embodiment further provides a locking member 164 to secure the pivot members 146 to prevent undesirable movement of the pivot members 146, such as during transport of the adjustable row arch applicator 118.

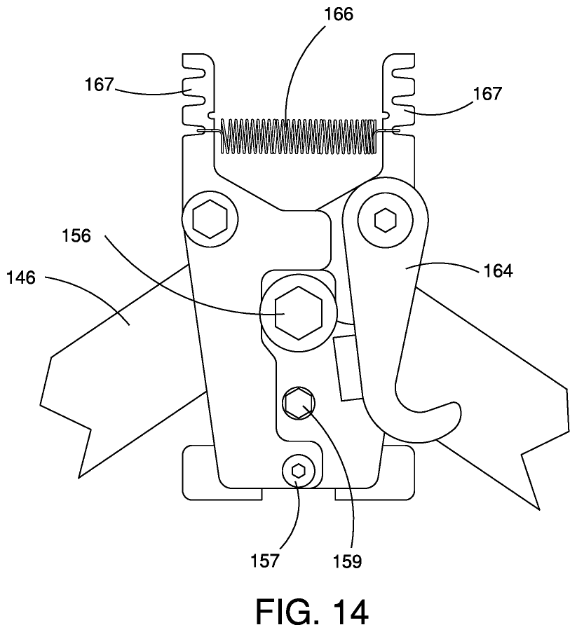

Turning to FIG. 15, the interior of a self-adjusting pivot 154 of the present invention is shown. The figure shows the top of a pivot member 146. Also shown is a bridge 168 that connects the two pivot members 146. The second pivot member is not shown in FIG. 15, but in the illustrated embodiment, the two pivot members 146 and bridge 168 are symmetrical and form one continuous component. The applicator pivot point 156 extends through the bridge 168. In some embodiments, the resistance between the two pivot members 146 is balanced at the applicator pivot point 156. Force in either direction, which will be discussed in detail below, will cause the pivot members 146 to respond and move accordingly. In the illustrated embodiment, the pivot members 146 and bridge 168 create a pendulum that moves in an arcuate path.

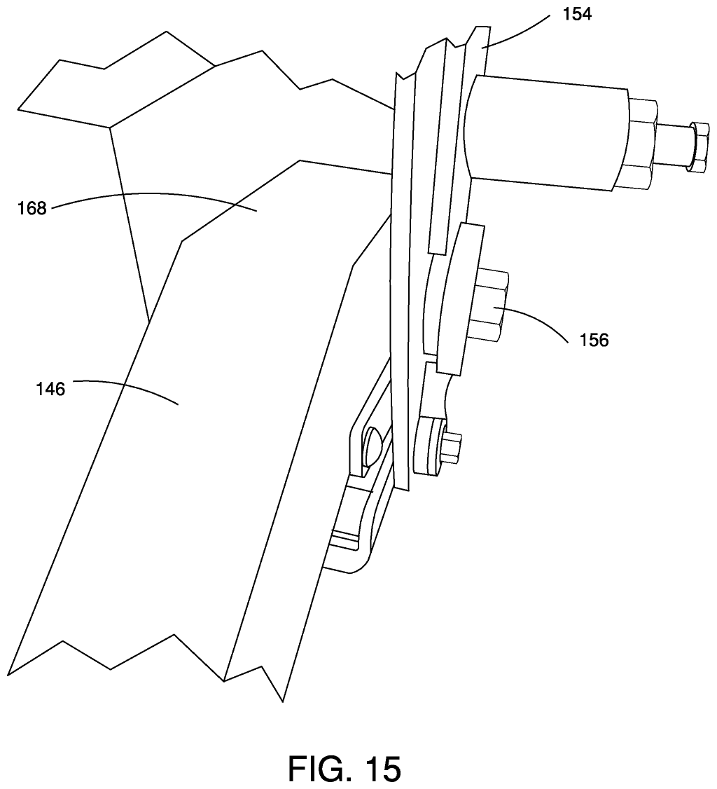

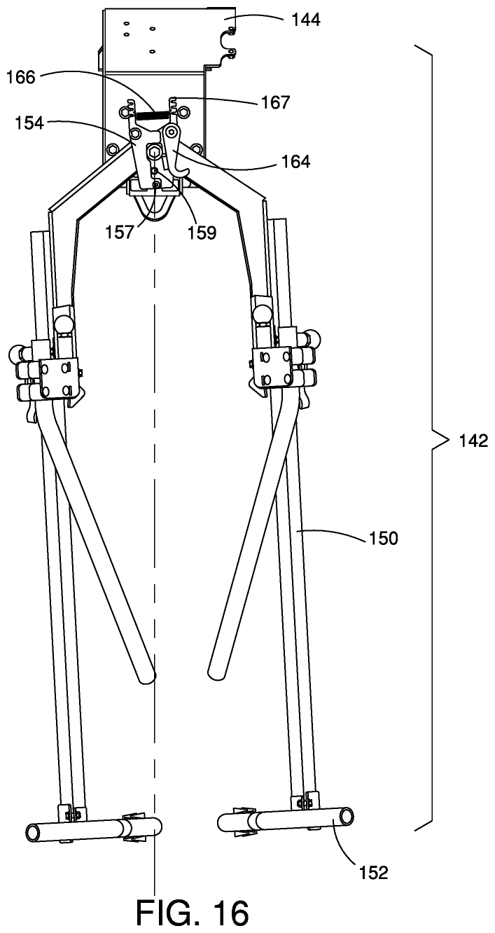

FIGS. 16-17 illustrate operation of a head 142 of an adjustable row arch applicator 118 of one embodiment of the present invention. The head 142 may include a bracket 144 attached to a self-adjusting pivot 154. The self-adjusting pivot 154 may further include at least one locking member 164. The self-adjusting pivot 154 includes a biasing member, such as a spring 166. The spring 166 is attached to two biasing member prongs 167 to enable the operation of the biasing member. The locking member 164 may be in the form of an immobilizing latch 169. In FIG. 16, the self-adjusting pivot 154 is tilted towards the right relative to the balanced position when viewed from the front, movement which would have been actuated when the guide members 152 came into contact with a plant that was to the right of center. FIG. 17, on the other hand, illustrates the self-adjusting pivot 154 tilted towards the left when viewed from the front, movement which would have been actuated when the guide members 152 came into contact with a plant that was to the left of center. More specifically, in the illustrated embodiment, the biasing member prongs 167 are both connected to the biasing prong pivot point 157, as well as to the spring 166. Accordingly, when the pivot members 146 are shifted to the right as seen in FIG. 16, the actuating pin 159 comes into contact with the right biasing member prong 142, causing the right biasing member prong 167 to move away from the left biasing member prong 167, tensioning the spring 166. When the force acting on the guide members 146 is no longer present, the spring 166 will contract and force the right biasing member prong 167 back towards the left prong 167, returning the pivot members 146 to their neutral position. In FIG. 16, the applied force moves the actuating pin 159 into contact with the left biasing member prong 167. This creates a similar effect, where the spring 166 will return both itself and the pivot members 146 to a neutral position after the applied force is no longer present. The head further comprises one or more pivot members 146 connected to the self-adjusting pivot 154. The adjustable length members 150 may be adjusted by a pin adjuster 160.

The applicator, such as an adjustable row arch applicator 118 can be used to apply products to any species of crop plant that can be accommodated by the device to which the applicator is affixed. In general, the applicator is designed for use on plants that are grown in rows. However, different embodiments of the applicator device can be adapted to operate in any environment including, but not limited to, ideal or target outdoor growing environments, off-season environments, or controlled environments (e.g. shade/glass/green/hoop houses, growth chambers, vertical farming facilities, hydroponic facilities, aeroponic facilities etc.).

The precision particulate matter metering apparatuses of the invention can be affixed to a variety of vehicles allowing it to travel across rows of plants. The metering apparatuses can be mounted to manual delivery vehicles or robots that might be used in indoor environments or smaller plot sizes. The apparatus can also be mounted to field driven machinery.

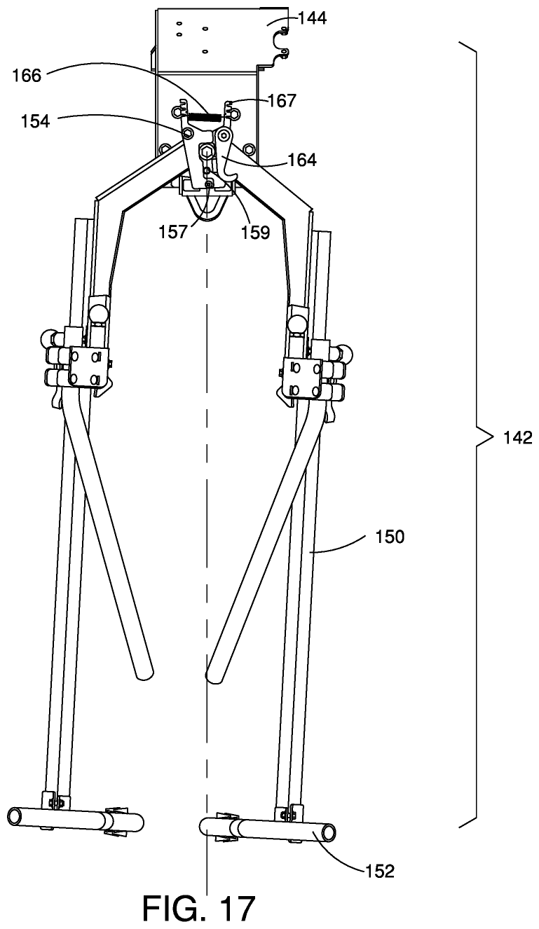

Turning to FIG. 18, another embodiment of a plurality of precision metering apparatuses 100 are shown wherein the compression device is a piston 178 contained within a solid particle material receptacle 176 attached to piece of field machinery 102 via support network 104. In the shown embodiment, six apparatuses 100 are attached to a piece of field machinery 102 for the purpose of clarity. The precision metering apparatuses 100 of the shown embodiment comprise a vertical compression tube 109 connected to a distribution chamber 112. Each distribution chamber 112 has a material outlet 188 which feeds one side of an applicator 117 such as an adjustable row arch applicator 118 via a distribution tube 122.

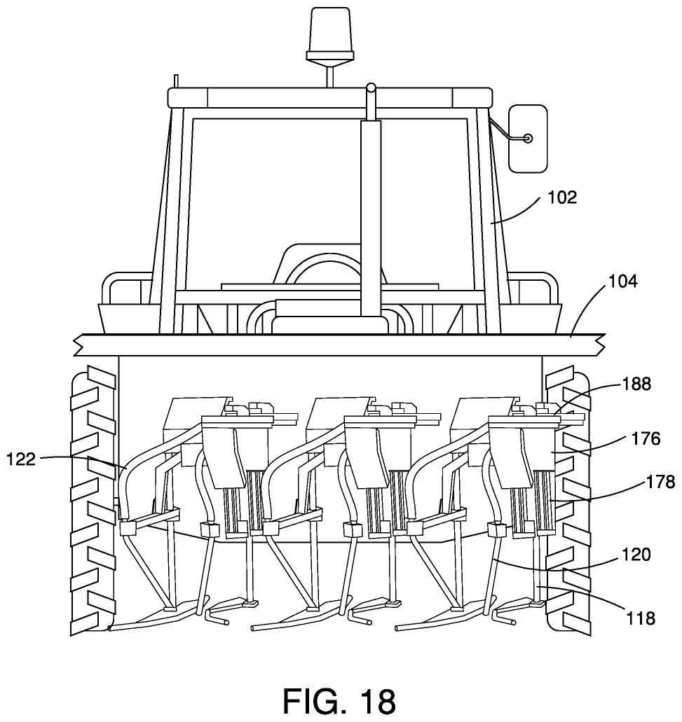

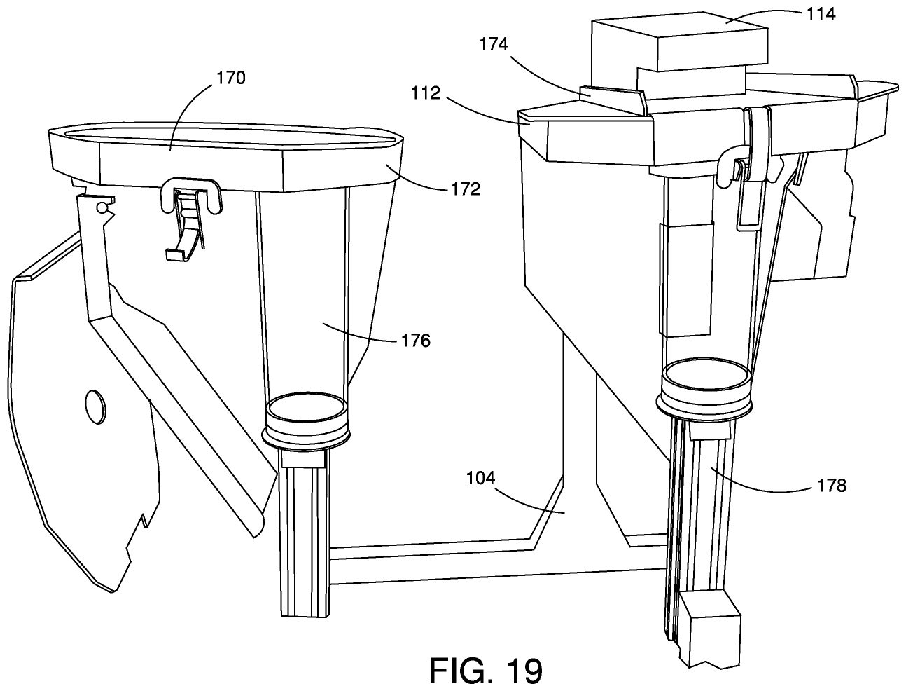

Turning to FIG. 19, another view of the embodiment of FIG. 18 is shown. The precision metering apparatus 100 shown in FIG. 19 may include a blower fan 114 and a shaving mechanism attached to a distribution chamber 112. In the embodiment shown in FIG. 18, the shaving mechanism comprises an impeller 170 (seen in more detail in FIGS. 20 -22), and the distribution chamber 112 comprises an impeller housing 172, and an impeller housing cover 174. In the apparatus 100 shown on the left, the impeller housing cover 174 is shown off and to the side of the apparatus 100. The embodiment further comprises a compression tube 109 for the storage and compression of pollen or other solid particulate material 196. In the illustrated embodiment, the compression tube 109 is a solid particulate material receptacle 176. The solid particulate material receptacle 176 further includes a piston 178 to compress and dispense the pollen or other solid particulate material 196. In FIG. 19, the impeller housing cover 174 has been removed or opened from the precision pollen metering apparatus 100 on the left. As the blower fan 114 in the illustrated embodiment sits atop the impeller housing cover 174, the blower fan 114 is also moved when the cover 174 is in the open position. Further figures will show interior features of the lefthand, open apparatus 100, which will be discussed below. The solid particulate receptacle 176 may be any shape or size desired by a user. In the illustrated embodiment the receptacle 176 takes the form of a canister that is a cylinder. The receptacle 176 is in operational engagement with a piston 178. The piston 178 may be any shape or size, but preferably is complimentary in shape and size to the receptacle 176.

Turning to FIG. 20, another view of the embodiment of FIG. 19 is shown. The impeller housing cover 174 and blower fan 114 of an alternative embodiment of the present invention is shown. In the illustrated embodiment, the fan 114 is located on top of the cover 174. However, the fan 114 may be located in any position wherein it is able to provide pressure, such as positive pressure, within the distribution chamber 112 as described herein below.

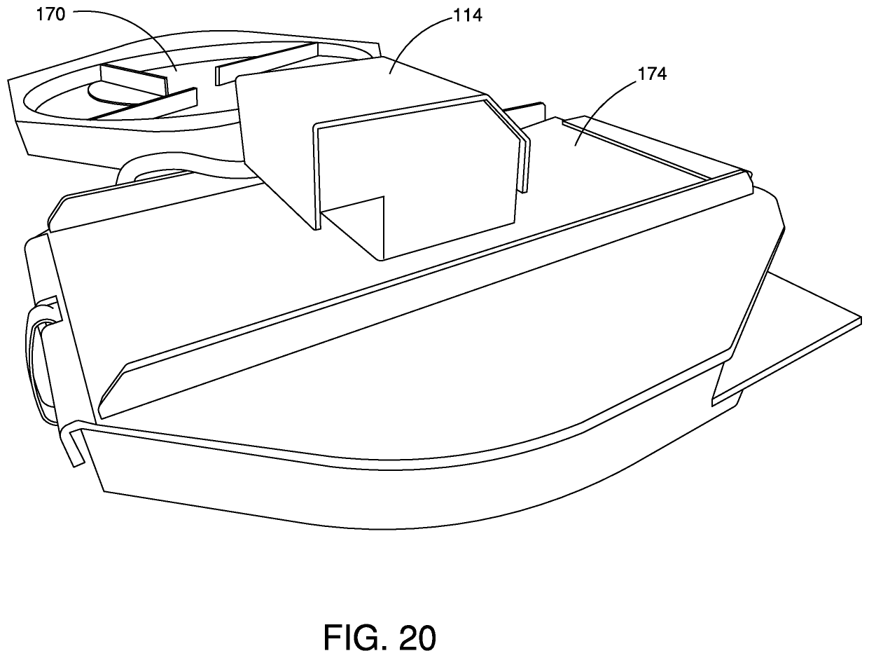

FIG. 21 shows the open distribution chamber 112 of the apparatus 100 of FIG. 19. Seen is the impeller 170 located within the distribution chamber 112, which is created by the impeller housing 172. The impeller cover 174 has been opened and is not shown. Also shown is an inlet aperture 180 located directly above the solid particulate receptacle 176. The impeller 170 may have one or more projections 182, such as a blade. In preferred embodiments, the impeller 170 includes a plurality of projections 182. One or more of the projections 182 may have an edge 184. The edge 184 may be raised relative to the surface 186 of the projection 182. The impeller 172 is operationally attached to a motor (not shown) that results in rotational movement of the impeller 170. Also shown in FIG. 21 is a material outlet 188. The material outlet 188 is connected to an applicator device, such as an adjustable row arch applicator 118 by distribution tubes 122.

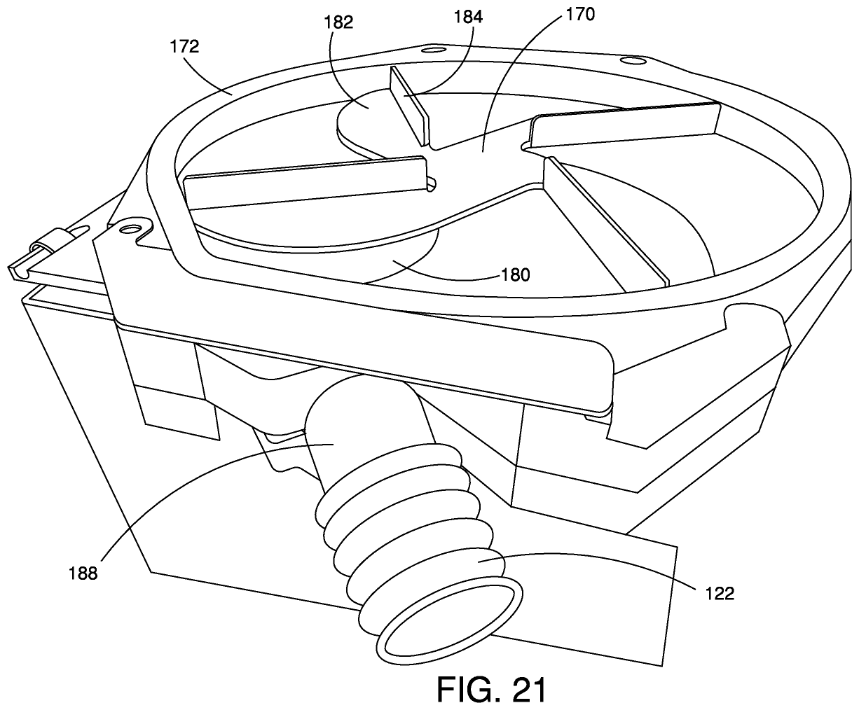

FIG. 22 provides another view of the open distribution chamber 112 of the embodiment of FIG. 19 showing a magnified view of the impeller projection 182 and inlet aperture 180. Shown is a portion of the impeller 170 including an impeller projection 182. The impeller projection 182 includes an edge 184. The shown projection 182 is over the inlet aperture 180. The inlet aperture 180 is preferably located directly above the receptacle 176 so that material may move from the receptacle 176 into the distribution chamber 112. Also shown is a material outlet 188.

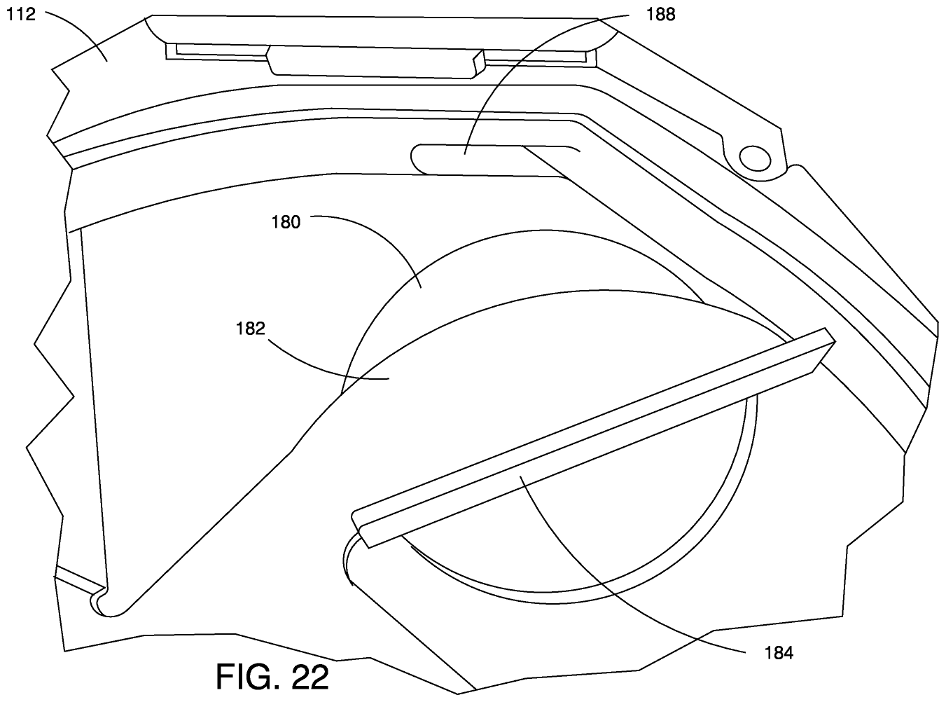

Yet another view of the open distribution chamber 112 of the example embodiment is found in FIG. 23. Shown is the impeller 170 including four impeller projections 182. The projections 182 each have an edge 184 which is preferably raised with respect to the impeller surface 186. The distribution chamber 112 includes a circular side 190, although any shape may be used. Preferably the shape and size of the chamber 112 are complimentary to the shape and size of the impeller 170. The distribution chamber 112 also includes a bottom 192. In the illustrated embodiment, the size of the distribution chamber 112, including the side 190 and bottom 192 are close in size to the diameter of the impeller 170. Also shown are the inlet aperture 180 and material outlet 188. The material outlet 188 is connected to the distribution tube 122.

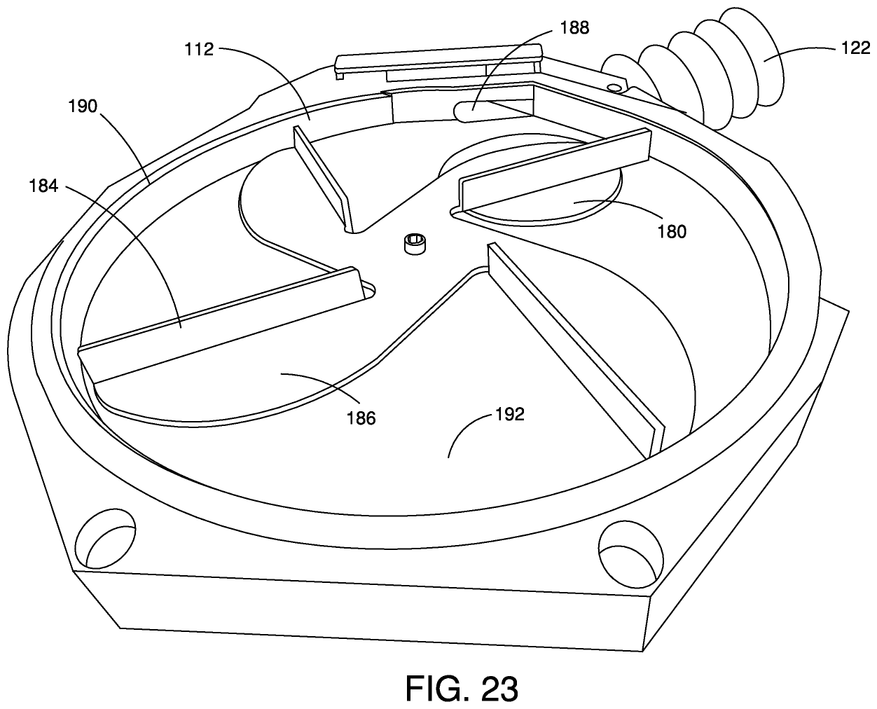

FIG. 24 shows the solid particulate material receptacle 176 to further illustrate the shape and configuration. In the illustrated embodiment, the receptacle 176 is cylinder shaped; however, any shape or size receptacle 176 may be used. Also shown is the piston 178. The piston 178 is preferably of a shape and size that is complimentary to the shape and size of the receptacle 176 to carry out the function described herein below. The piston 178 is in operational engagement with the bottom 194 of the solid particle material receptacle 176. The piston 178 is configured to push the bottom 194 through the receptacle 176. As such, the bottom 194 is configured to be movable within the receptacle 176.

The operation of the embodiment of FIG. 18 of a metering apparatus 100 serves to precisely meter and distribute solid particulate material 196 such as pollen. Solid particulate material 196 is placed into the solid particulate receptacle 176 by any means known now or in the future. In one example, solid particulate material 196 is manually loaded into the receptacle 176 for dispersal. In one embodiment, the receptacle 176 is removable from the apparatus 100. The receptacle may be filled with solid particulate material 196 via a funnel. The receptacle 176 containing solid particulate material 196 may then be put into place in the device 100. In other embodiments, solid particulate material 196 and/or receptacles 176 of same may be loaded automatically.

The piston 178 is operationally connected to a motor-driven actuator that causes the piston to raise and lower within the receptacle 176. Any means known now or in the future may be used to power movement of the piston 178, including but not limited to hydraulic power, pneumatic power, and/or electric driven power, such as stepper, servo, axial, cog driven, etc. When the piston 178 is raised within the receptacle 176, it pushes on the receptacle bottom 194 which is movable up into the receptacle 176 forming a compression zone 134. Therefore, the solid particulate material 196 is pushed up and out of the top of the receptacle 176. The top of the receptacle 176 aligns with the inlet aperture 180. As such, the material is pushed through the inlet aperture 180 and into the distribution chamber 112. During operation, the impeller 170 rotates. Preferably, the impeller 170 rotates continuously throughout operation. In the illustrated embodiment, the impeller 170 is powered by a motor (not shown) but may be powered by any means known in the art now or in the future, including but not limited to hydraulic power, pneumatic power, and/or electric driven power, such as stepper, servo, axial, cog, etc. As the solid particulate material 196 moves into the impeller chamber, the rotating impeller projections 182 operate to move one or more portions of the material away from the end of the compression tube 109 and inlet aperture 180 into the distribution chamber 112. Said another way, the impeller 170 shaves material off as it is presented through the inlet aperture 180. This may cause the solid particulate material 196 to form a cloud in the chamber.

The blower fan 114 operates to create pressure within the distribution chamber 112, preferably positive pressure. In addition, or alternatively, other means of creating pressure, such as positive pressure, known in the art now or in the future may be used including, but not limited to, an impeller, pump, vacuum pneumatics, or hydraulics. The positive pressure forces the material that has been moved into the distribution chamber 112 into the material outlet 188. The material then moves into a distribution tube 122. In the preferred embodiment, a distribution tube 122 distributes material directly to a plant through an adjustable row arch applicator 118. The distribution tubes 122 and/or applicator tips 120 may be configured to customize the pattern, direction, and/or velocity of the material as it exits the system. The distribution tubes 122 may have an adjustable airflow, which may allow the material 196 in the tubes 122 to flow at a prescribed rate (distance over time). The applicator tips 120 may propel and/or target the material out of the device. As one having at least ordinary skill in the art will recognize, other configurations may deliver material without departing from the scope of the invention. Operation of the piston 178 in combination with the impeller 170 serves to precisely meter the amount of solid particulate material 196 that is delivered by the device 100.

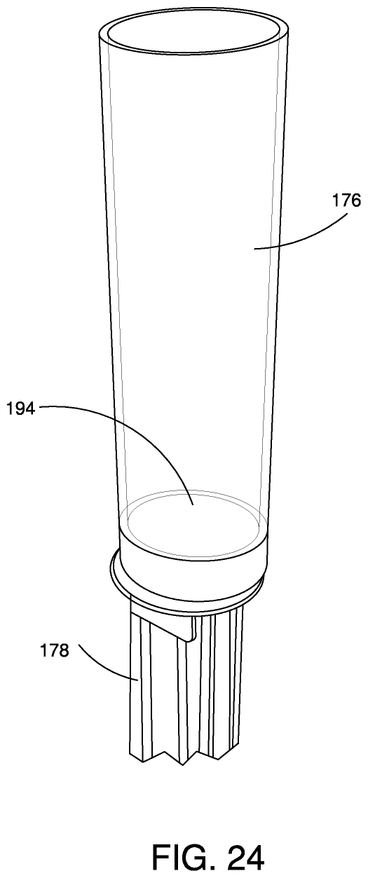

FIGS. 24-28 illustrate the movement of particulate material 196 though a first embodiment of the present invention. In FIG. 25, disaggregated particulate material 196 is shown in the auger chute 108 of the shown embodiment. Though the auger chute 108 is shown in a cutaway view to see the particulate material 196, the particulate material 196 would also be contained in the storage bin 106 above the auger chute 108. Also shown is the agitator 136 and corresponding agitator motor 138 which help the particulate material move through to the compaction tube 109.

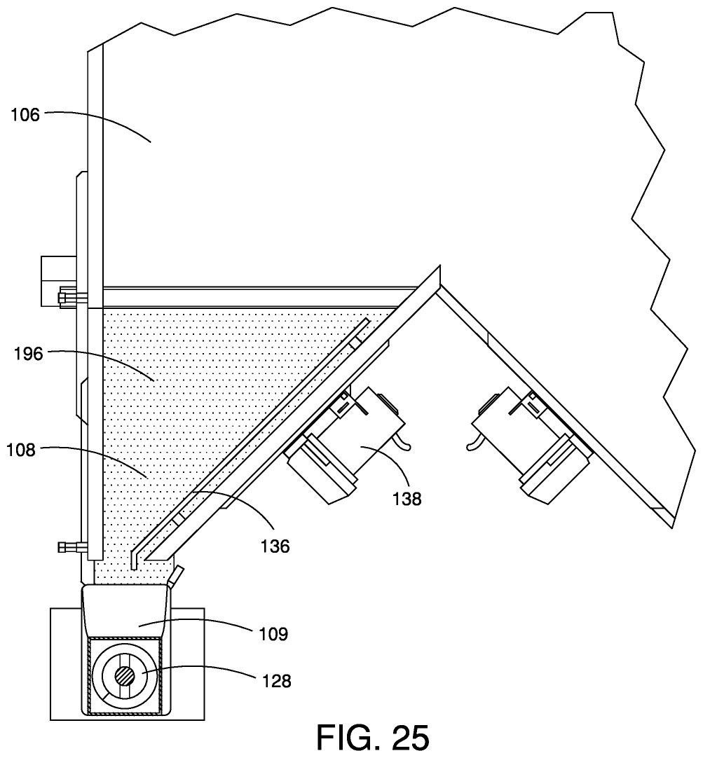

Shown in FIG. 26 is a cutaway view of an embodiment of a dosing apparatus 107 of the present invention showing the compression of disaggregated particulate material 196 into a cohesive cylindrical disk or puck 111. Also shown is the shaftless auger 124. The auger 124 includes an auger position adjustment mechanism 125 which is capable of adjusting the position of the auger 124 laterally within the auger tube 110 to either increase or decrease the size of the compression zone 134. Below the auger tube 110 is shown an auger position adjustment motor 127 that operates the auger position adjustment mechanism 125 to increase or decrease the compression zone 134 by adjusting the position of the shaftless auger 124 within the auger tube 110. In the shown embodiment, the auger position adjustment mechanism 125 is a screw adjuster 129 connected to a motor 127, where the rotation of the screw 129 by the motor 127 will move the auger 124 along the screw 129 either forwards or backwards. At the end of the auger tube 110 a dosing plate 140 is shown which shaves extruded pollen or other particulate material 196 off of the puck 111 formed in the compression zone 134.

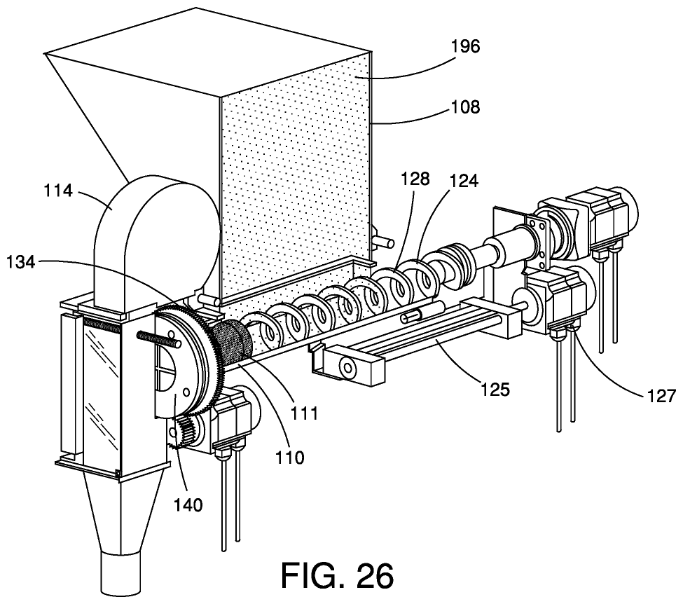

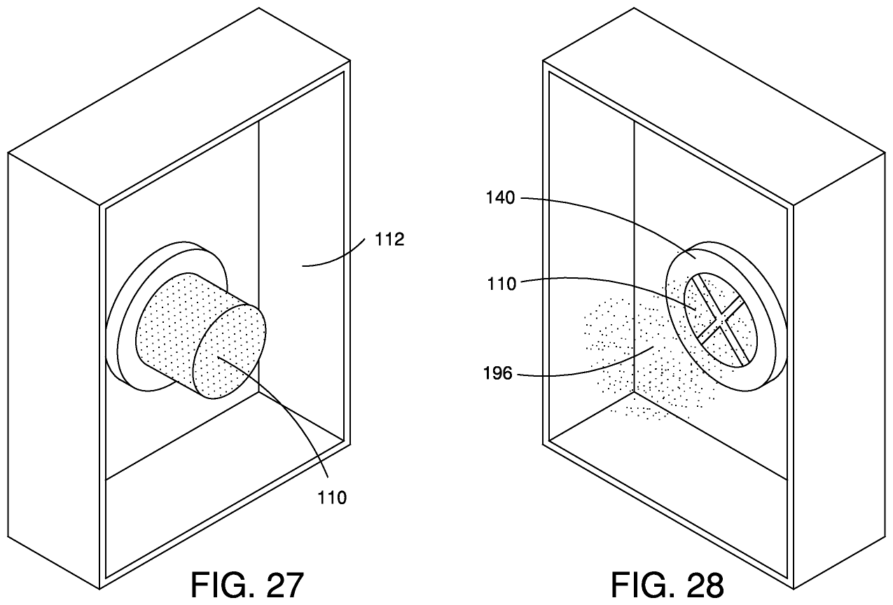

Shown in FIG. 27 is a cutaway view of a distribution chamber 112 of the present invention with the dosing plate 140 removed to show the extrusion of a puck 111 of particulate material 196. Shown in FIG. 28 is a similar cutaway view of a distribution chamber 112 with the dosing plate 140. The dosing plate is shown shaving particulate material 196 off of the extruded puck 111, where it is exposed to air from the blower fans 114 (not shown). The shaved particulate material 196 is directed by gravity and the blower fans 114 towards the distribution funnels.

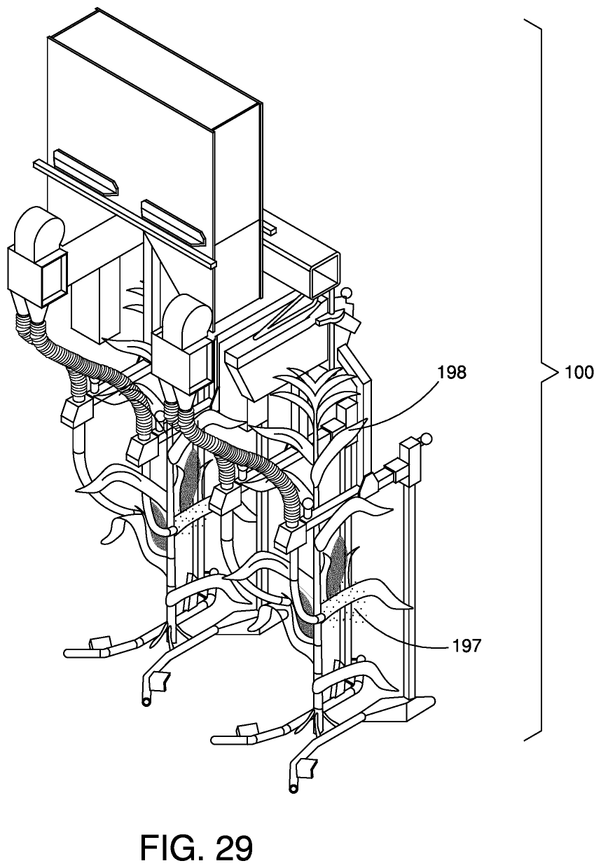

Shown in FIG. 29 is a metering apparatus 100 of the present invention dispersing particulate material 196 in the form of a particulate material cloud 197 onto a tall row crop 198. For clarity, FIG. 29 shows the metering apparatus 100 apart from the field equipment 102 to which it would be attached in normal operation.

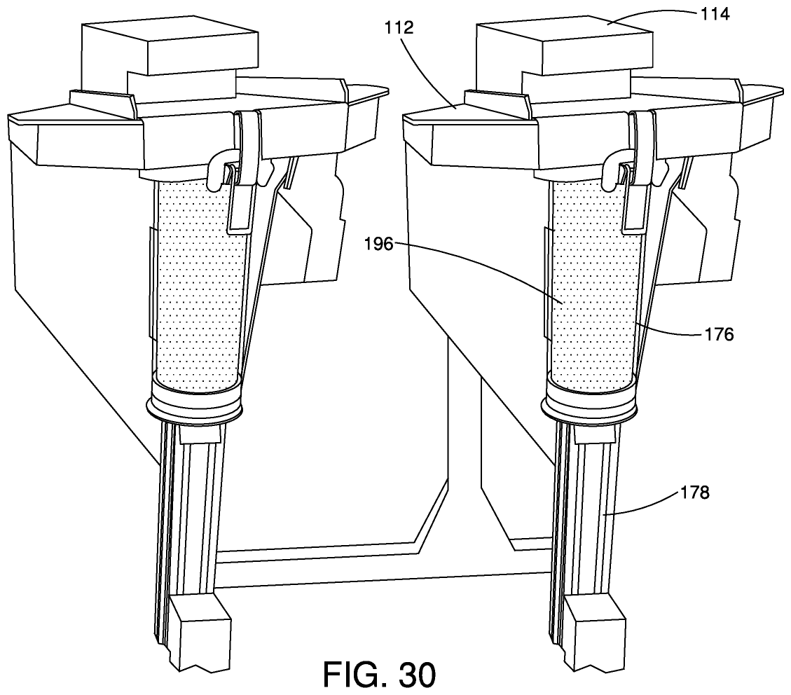

FIGS. 30-33 illustrate the movement of particulate material though a second embodiment of the present invention. The precision metering apparatus 100 shown in FIG. 30 includes a blower fan 114 attached to a distribution chamber 112. The embodiment further comprises a compression tube 109 for the storage and compression of pollen or other solid particulate material 196. In the illustrated embodiment, the compression tube 109 is a solid particulate material receptacle 176. The solid particulate material receptacle 176 further includes a piston 178 to compress and dispense the pollen or other solid particulate material 196. The solid particulate receptacles 176 are shown filled with solid particulate material 196. The receptacle 176 is in operational engagement with a piston 178. The piston 178 may be any shape or size, but preferably is complimentary in shape and size to the receptacle 176.

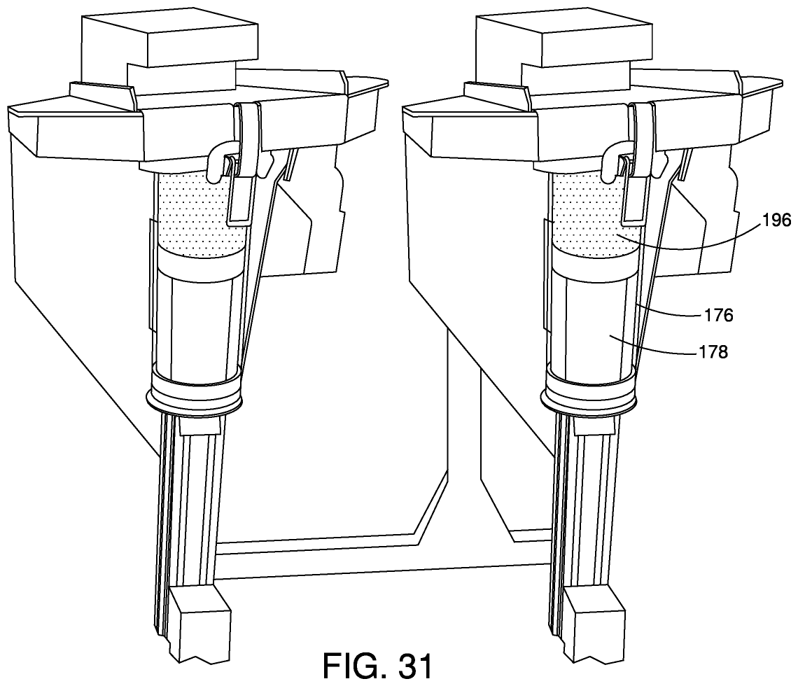

Shown in FIG. 31 is another view of the embodiment of FIG. 30 showing the metering apparatus 100 dispensing particulate material 196 by raising the piston 178 to push the particulate material 196 into the distribution chamber 112.

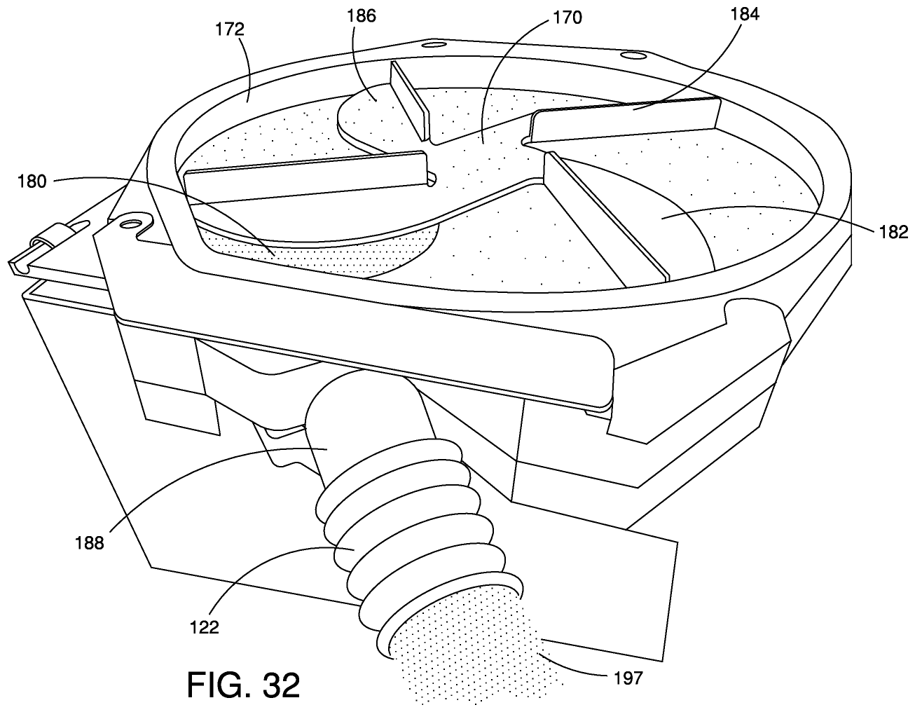

Shown in FIG. 32 is the distribution chamber 112 of the apparatus 100 of FIG. 31. A cover 174 which would normally be installed during operation is not pictured in order to more easily view the operation of the apparatus 100. Shown is an impeller 170 located within the distribution chamber 112, which is created by the impeller housing 172. Also shown is an inlet aperture 180 located directly above the solid particulate receptacle 176. The impeller 170 may have one or more projections 182, such as a blade. In preferred embodiments, the impeller 170 includes a plurality of projections 182. One or more of the projections 182 may have an edge 184. The edge 184 may be raised relative to the surface 186 of the projection 182. The impeller 172 is operationally attached to a motor (not shown) that results in rotational movement of the impeller 170. Also shown in FIG. 32 is a material outlet 188. The material outlet 188 is connected to an applicator device, such as an adjustable row arch applicator 118 by distribution tubes 122. In the illustrated embodiment, particulate material 196 is being pushed into the impeller chamber 172 where is shown being shaved off by the impeller projections 182. After being shaved, the particulate material travels through the distribution chamber 112 and out through the distribution tubes 122 as a particulate material cloud 197.

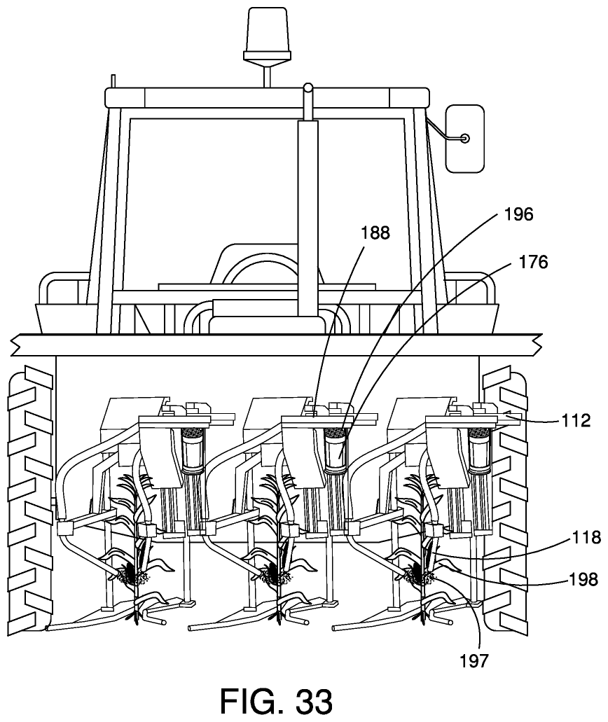

Shown further in FIG. 33, is the distribution of a particulate material cloud 197 onto a tall row crop 198. Only one pair of precision metering apparatuses 100 are shown in use for clarity, though in normal operation, one or more pairs of precision metering apparatuses 100 would be used in conjunction to apply solid particulate material to a plurality of tall row crops 198 at the same time. Also shown is a piston 178 contained within a solid particle material receptacle 176 attached to piece of field machinery 102 via support network 104. In the shown embodiment, eight apparatuses 100 are attached to a piece of field machinery 102. The solid particulate material receptacles 176 are shown containing solid particulate material 196. The precision metering apparatuses 100 of the shown embodiment comprise a vertical compression tube 109 connected to a distribution chamber 112. Each distribution chamber 112 has a material outlet 188 which feeds one side of an applicator 117 such as an adjustable row arch applicator 118 via a distribution tube 116. In the shown embodiment, solid particulate material 196 that has been shaved off in the distribution chamber 112 is transported through the distribution tubes 120 by the blower fan 114. The particulate material 196 is carried to the tall row crow 198 through the applicator tips 120, and is shown exiting the applicator tips 120 as a particulate material cloud 197.

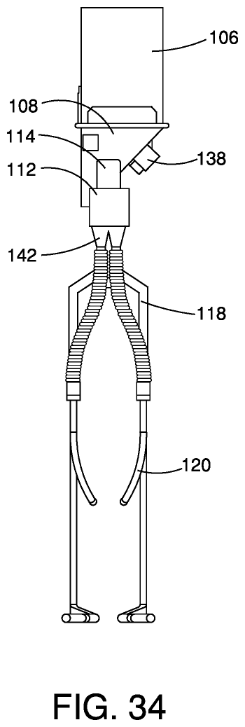

Shown in FIG. 34 is another embodiment of the present invention wherein the storage bin 106 is not shared between two separate auger chutes 108, and is instead fixed to only one auger chute 106. The auger chute 106 is connected to a compaction tube 109 (not shown), which feeds into a distribution chamber 112. On top of the distribution chamber 112 is a blower fan 114. The distribution chamber 112 further includes two distribution funnels 142 connected to the bottom of the distribution chamber 112. The distribution funnels 142 are connected to the applicator tips 120 by distribution tubes 122.

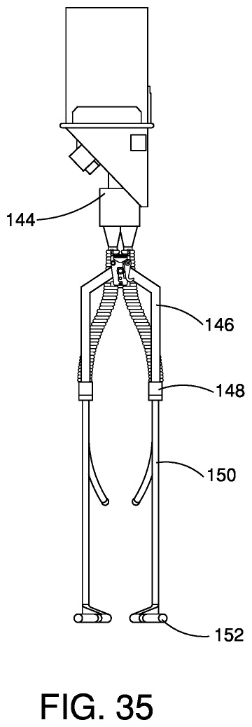

Shown in FIG. 35 is a rear view of embodiment of FIG. 34, showing the applicator 117 in more detail. The bracket 144, connects the applicator to a support network 104 (not shown). Also shown are two pivot members 146, each connected to a brace 148. Each brace 148 is connected to a distribution tip 120 and an adjustable length leg member 150. Attached to the end of the adjustable length leg members is a guide member 152.

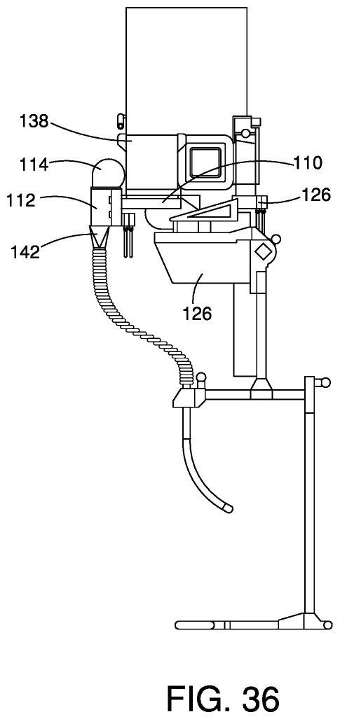

Depicted in FIG. 36 is a side view of the embodiment of FIG. 34. An auger tube 110 is shown, along with an auger motor 126. Also shown is an auger position adjustment mechanism 125 connected to an auger position adjustment motor 127. Also shown is a distribution chamber 112 connected to the auger tube 110. A blower fan 114 is shown mounted above the distribution chamber 112. Also shown is a distribution funnel 142 connected the distribution chamber 112 to an applicator tip 120 by a distribution tube 122.

### EXAMPLES

### Example 1

Experiments were performed to measure the impact of different embodiments of the invention on pollen viability. A total of nine experiments were performed by mounting different embodiments of the invention to a piece of field equipment and using them to apply pollen. The amount of actual pollen applied was varied in the experiments from 1-2 liters per acre. Additionally, the speed of the field equipment was varied in the experiments from 4.5-6 miles per hour. Pollen used was mixed with an additive at a ratio of one part pollen to five parts additive. Also performed was a negative control, where no pollen was applied, as well as a test involving the hand application of pure pollen with no additive. Groupings that do not share a letter are significantly different. The results are shown below in Table 1A.

TABLE 1A
Results of experiments testing the effectiveness of invention
in metering live pollen while maintaining viability.

| | Number | Mean Kernel | |
|---|---|---|---|
| Applicator/Tubes | of Ears | Count | Grouping |
| Dosing Plate Auger | 650 | 94 | A |
| with Long Tubes | | | |
| Dosing Plate Auger | 747 | 92 | A |
| with Short Tubes | | | |
| Piston Impeller with | 922 | 77 | B |
| Short Tubes | | | |
| Squirrel Cage Auger | 375 | 45 | C |
| with Long Tubes | | | |
| Squirrel Cage Auger | 337 | 61 | D |
| with Short Tubes | | | |

Shown below in Table 1B is the normalized kernel count for the various embodiments based on the results of the experiments. The normalized kernel count was computed by taking the observed kernel count for each tested embodiment, subtracting the observed kernel count for the negative control, and then dividing that number by the kernel count for the hand-applied pollen minus the kernel count for the negative control.

TABLE 1B
Results of experiments testing the effectiveness of invention
in metering live pollen while maintaining viability
normalized with respect to negative control

| | | Mean Normalized | |
|---|---|---|---|
| Applicator/Tubes | Number of Ears | Kernel Count | Grouping |
| Dosing Plate Auger | 650 | 0.28 | A |
| with Long Tubes | | | |
| Dosing Plate Auger | 747 | 0.26 | A/B |
| with Short Tubes | | | |
| Piston Impeller with | 922 | 0.26 | A/B |
| Short Tubes | | | |
| Squirrel Cage Auger | 375 | 0.23 | B |
| with Long Tubes | | | |
| Squirrel Cage Auger | 337 | 0.28 | A/B |
| with Short Tubes | | | |

Shown below in Table 2A is the mean kernel count for selected experiments. Specifically, experimental runs with results that had all treatments averaging under 50 kernel count were not included in the data set, as the low kernel count across the board indicated that the results could have been affected by extraneous environmental conditions.

TABLE 2A
Results of selected experiments testing the effectiveness of
invention in metering pollen while maintaining viability.

| | Number | Mean Kernel | |
|---|---|---|---|
| Applicator/Tubes | of Ears | Count | Grouping |
| Dosing Plate Auger | 382 | 140 | A |
| with Long Tubes | | | |
| Dosing Plate Auger | 392 | 149 | A |
| with Short Tubes | | | |
| Piston Impeller with | 474 | 125 | B |
| Short Tubes | | | |
| Squirrel Cage Auger | 202 | 55 | C |
| with Long Tubes | | | |
| Squirrel Cage Auger | 164 | 95 | D |
| with Short Tubes | | | |

Shown below in Table 2B is the normalized kernel count for the various embodiments in selected experiments. The normalized kernel count was computed by taking the observed kernel count for each tested embodiment, subtracting the observed kernel count for the negative control, and then dividing that number by the kernel count for the hand-applied pollen minus the kernel count for the negative control. Additionally, Experimental runs with results that had all treatments averaging under 50 kernel count were not included in the data set, as the low kernel count across the board indicated that the results were affected by extraneous environmental conditions.

TABLE 2B
Results of selected experiments testing the effectiveness of
invention in metering pollen while maintaining viability
normalized with respect to the negative control.

| | | Mean Normalized | |
|---|---|---|---|
| Applicator/Tubes | Number of Ears | Kernel Count | Grouping |
| Dosing Plate Auger | 382 | 0.34 | A |
| with Long Tubes | | | |
| Dosing Plate Auger | 392 | 0.37 | A |
| with Short Tubes | | | |
| Piston Impeller with | 474 | 0.30 | B |
| Short Tubes | | | |
| Squirrel Cage Auger | 202 | 0.12 | C |
| with Long Tubes | | | |
| Squirrel Cage Auger | 164 | 0.23 | D |
| with Short Tubes | | | |

As can be seen from the table, the dosing plate with short distribution tubes performed the best at maintaining pollen integrity. Accordingly, for embodiments of the present invention designed to distribute viable pollen, the dosing plate design would be preferable due to the increased seed set in comparison to some of the alternative embodiments. However, one having at least ordinary skill in the art would recognize that all embodiments showed at least some efficacy in distributing pollen to receptive plants while maintaining viability of said pollen.

Although various representative embodiments of this invention have been described above with a certain degree of particularity, one having at least ordinary skill in the art could make numerous alterations to the disclosed embodiments without departing from the spirit or scope of the inventive subject matter set forth in the specification and claims. In some instances, in methodologies directly or indirectly set forth herein, various steps and operations are described in one possible order of operation, but one having at least ordinary skill in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to the embodiments outlined above, various alternatives, modifications, variations, improvements and/or substantial equivalents, whether known or that are or may be presently foreseen, may become apparent to one having at least ordinary skill in the art. Listing the steps of a method in a certain order does not constitute any limitation on the order of the steps of the method. Accordingly, the embodiments of the invention set forth above are intended to be illustrative, not limiting. Changes may be made in form and detail without departing from the spirit and scope of the invention. Therefore, the invention is intended to embrace all known or earlier developed alternatives, modifications, variations, improvements, and/or substantial equivalents.

1. A precision metering and delivery apparatus for the precise distribution of particulate material via compression and metered shaving of said particulate material, said precision metering apparatus comprising:
a. a delivery system,
b. a dosing apparatus, and
c. a storage device.

2. The precision metering and delivery apparatus of claim 1 wherein said precision metering and delivery apparatus is mounted on field equipment.

3. The precision metering and delivery apparatus of claim 1 further comprising a compression device.

4. The precision metering and delivery apparatus of claim 3 wherein said compression device is a shaftless auger.

5. The precision metering and delivery apparatus of claim 3 wherein said compression device is a piston.

6. The precision metering and delivery apparatus of claim 1 wherein said dosing apparatus comprises a rotating dosing plate.

7. The precision metering and delivery apparatus of claim 1 wherein said dosing apparatus comprises a rotating impellor.

8. The precision metering and delivery apparatus of claim 4 wherein said shaftless auger comprises:
a. Shaftless flighting predominately coextensive with said compression tube; and
b. a position adjustment mechanism.

9. The precision metering and delivery apparatus of claim 8 further comprising an auger chute between said storage bin and said compression device.

10. The precision metering and delivery apparatus of claim 1 wherein said delivery system comprises at least one blower fan and at least one applicator.

11. A precision pollen metering and delivery apparatus for the precise distribution of viable pollen via compression and metered shaving of said pollen, said precision pollen metering and delivery apparatus comprising:
a. a compression device,
b. a delivery system,
c. a dosing apparatus,
d. a compression tube,
e. a compression zone between the terminal end of said compression device and the terminal end of said compression tube, and
f. at least one applicator.

12. The precision pollen metering and delivery apparatus of claim 11 wherein said applicator further comprises at least one guide member and at least one applicator tip.

13. The precision pollen metering and delivery apparatus of claim 12 wherein said applicator is an arch applicator having at least one applicator tip on each side of said arch.

14. A method of distributing viable pollen to plants comprising:
a. compressing disaggregated pollen into a semisolid shape;
b. shaving said pollen from said semisolid shape at a consistent rate; and
c. dispensing said shaved pollen to the stigma of said plant.

15. The method of claim 14 wherein said compression step occurs via a shaftless auger.

16. The method of claim 14 wherein said compression step occurs via a piston.

17. The method of claim 14 wherein said shaving step occurs via a dosing plate.

18. The method of claim 14 wherein said shaving step occurs via an impeller.

19. The method of claim 14 wherein said dispensing step occurs via an air delivery system.

20. The method of claim 14 wherein said semisolid shape is a circular puck.
